(19) United States
(12) Patent Application Publication
(10) Pub. No.: US 2016/0054409 A1
(43) Pub. Date: Feb. 25, 2016

(54) FMRI-BASED NEUROLOGIC SIGNATURE OF PHYSICAL PAIN

(71) Applicants: Tor WAGER, (US); Martin LINDQUIST, (US)

(72) Inventors: Tor WAGER, Boulder, CO (US); Martin LINDQUIST, Baltimore, MD (US)

(21) Appl. No.: 14/781,981

(22) PCT Filed: Apr. 9, 2014
(86) PCT No.: PCT/US14/33538

§ 371 Date: Oct. 2, 2015

Related U.S. Application Data
(60) Provisional application No. 61/810,178, filed on Apr. 9, 2013.

(51) Int. Cl. G01R 33/48; A61B 5/00; G01N 33/49; A61B 5/055

(52) U.S. Cl. CPC G01R 33/4806; A61B 5/055; A61B 5/4824; A61B 5/4839; A61B 5/4848; G01N 33/4925; A61B 2576/026

(57) ABSTRACT

Described herein is a novel fMRI-based neurologic signature that predicts pain. Further described are methods for detecting pain, for diagnosing pain-related neuropathic conditions and for predicting or evaluating efficacy of an analgesic based on the neurologic signature.

### CROSS REFERENCE TO RELATED APPLICATION

This application claims the benefit of priority under 35 U.S.C. §119(e) to U.S. Provisional Patent Application Ser. No. 61/810,178, filed Apr. 9, 2013, which is incorporated herein by reference.

### GOVERNMENT SUPPORT

This invention was made with government support under grant numbers DA027794 and MH076136 awarded by the National Institutes of Health. The U.S. government has certain rights in the invention.

### FIELD OF THE INVENTION

The present invention generally relates to the use of fMRI technology to determine a neurological signature of physical pain.

### BACKGROUND OF INVENTION

Although biomarkers for medical conditions have proliferated over the past 50 years, objective assessments related to mental health have lagged behind. Physical pain is an affliction associated with enormous cognitive, social, and economic costs, but pain is not easy to ascertain. It is primarily assessed through self-report, an imperfect measure of subjective experience, which hampers diagnosis and treatment. The capacity to effectively report pain is limited in many vulnerable populations, such as the very old or very young, those with cognitive impairments, and those who are minimally conscious. Moreover, self-report provides a limited basis for understanding the neurophysiological processes underlying different types of pain, and thus a limited basis for targeting treatments to the underlying neuropathology.

Functional magnetic resonance imaging or functional MRI (fMRI) is an imaging procedure that measures brain activity by detecting associated changes in blood flow. This technique relies on the fact that cerebral blood flow and neuronal activation are coupled. When an area of the brain is in use, blood flow to that region also increases. For example, blood oxygen-level dependent (BOLD) fMRI exploits the different magnetic signals generated by oxyhemoglobin and deoxyhemoglobin to identify areas of the brain with high oxygen demand, indicating increased activity. By generating a number of images in quick succession, changes in activity in response to a given stimulus can be detected, thereby demonstrating the correspondence between the stimulus and the brain region(s) involved in the task. BOLD fMRI is now routinely used to measure regional cerebral blood flow (rCBF) in response to changes in neuronal activity. While application of fMRI in the context of pain is plausible, so far no reliable fMRI application to detect pain has been developed that has been demonstrated to be both sensitive and specific to pain (or any subtype of pain) within an individual person, in a manner validated across different MRI scanners.

Current approaches to pain assessment focus on a convergence of biological, behavioral, and self-reporting measures. Thus, there continues to be a need in the art for methods that are sensitive and specific to physical pain and can provide objective measurements of pain. This application addresses such needs.

### SUMMARY OF INVENTION

In one aspect, the invention provides a method of detecting pain in a subject, including applying a stimulus to the subject, measuring brain activity of the subject in response to the stimulus using functional Magnetic Resonance Imaging (fMRI) and generating a brain map of the subject representing the brain activity of the subject; and comparing the brain map of the subject to a neurologic signature map, wherein the neurologic signature map represents brain activity indicative of pain. The signature map preferably comprises a fMRI pattern that is at least 70% identical to the fMRI pattern shown in FIG. 1A. In other embodiments, the method includes applying the signature map to the brain map of the subject to provide a response value. In some embodiments, the method comprises analyzing similarities and dissimilarities between portions of the brain map of the subject and the corresponding portions of the signature map. In some embodiments, the method includes quantifying the pain in the subject based on the response value.

These methods may also include diagnosing a pain-related condition in the subject, wherein the condition is selected from the group consisting of hyperalgesia, allodynia, pain catastrophizing, fear of pain, chronic neuropathic pain, complex regional pain syndrome, reflex sympathetic dystrophy, post-stroke pain, inflammatory pain, and nociceptive pain.

These methods may also include the administration of an analgesic to the subject. The analgesic may be selected based on the comparison between the brain map of the subject and the signature map. The dosage of the analgesic may be selected based on the comparison between the brain map of the subject and the signature map.

In these methods, the comparing step may be performed by a computer.

In these methods the subject is preferably a human.

In these methods the stimulus may be application of heat to the subject.

These methods may include measuring another indicator of pain in the subject, such is a verbal or nonverbal indicator.

Another method of the invention includes administering the analgesic to a subject, applying a stimulus to the subject, measuring brain activity of the subject in response to the stimulus using fMRI and generating a brain map of the subject representing the brain activity of the subject, and comparing the brain map of the subject to a signature map indicative of pain to determine the difference between the brain map of the subject and the signature map, wherein the signature map represents brain activity indicative of pain, wherein the dissimilarity between the brain map of the subject and the signature map is indicative of the efficacy of the analgesic. In these methods, the signature map preferably comprises a fMRI pattern that is at least 70% identical to the fMRI pattern shown in FIG. 1A. In these methods, the analgesic may be administered before, after or concurrently with the stimulus.

A related method of the invention includes measuring brain activity of a subject using fMRI and generating a brain map of the subject representing the brain activity of the subject and comparing the brain map of the subject to a signature map to determine the functional connectivity or structural connectivity between the brain regions of the subject, wherein the signature map represents brain activity indicative of pain.

Another embodiment is an fMRI pattern that is at least 70% identical to the fMRI pain signature pattern shown in FIG. 1A.

Another embodiment is a method for verifying pain in a subject comprising detecting oxygen consumption and blood flow of a brain of the subject by using an fMRI, and comparing the oxygen consumption and blood flow in the brain to the fMRI signature pain pattern of FIG. 1A.

### BRIEF DESCRIPTION OF DRAWINGS

FIG. 1 shows the prediction of physical pain based on normative data from other individuals in Study 1 (Prediction of pain in new participants). FIG. 1A) The signature map: voxels in which activity reliably predicts pain. The map is thresholded (q<0.05 False Discovery Rate corrected) for display only; all weights were used in prediction. FIG. 1B) Signature response (y-axis) vs. pain intensity (x-axis) for heat, anticipation, and pain recall events. Signature response values were calculated by taking the dot-product of the signature pattern weights and parameter estimates from a standard, single-participant general linear model with regressors for each condition. The estimates shown are derived from cross-validation, so that signature weights and test data are independent. Receiver operating characteristic (ROC) plots showed the tradeoff between specificity (x-axis) and sensitivity (y-axis) when lines were produced using fitted curve, assuming Gaussian signal distributions. Pain/no-pain and forced-choice tests were analyzed. Forced-choice performance was at 100% for all conditions. Error bars show standard error of the mean (SEM). Abbreviations: ACC, anterior cingulate; CB: cerebellum, Fus, fusiform; INS, insula; Hy, hypothalamus; IFJ, inferior frontal junction; OG, occipital gyms, PAG, periaqueductal gray; PCC, posterior cingulate; SMA, supplementary motor area; SPL, superior parietal lobule, SMG, supramarginal gyms; Thal, thalamus. Directions: a, anterior; i, inferior; 1, lateral; m, middle; p, posterior; s, superior; v, ventral.

FIG. 2 shows the application of the fMRI signature of FIG. 1A to Study 2. FIG. 2A) Signature response (y-axis) across temperatures used in Study 2 (x-axis). Signature response was defined as the dot-product of the signature pattern weights from Study 1 and the activation maps for each temperature within each individual (error bars show within-participant SEM). The relationship increases with increasing temperature, as does pain report. Percentages indicate forced-choice classification sensitivity/specificity for adjacent temperatures, and reflect the proportion of participants in which the correct decision was made. FIG. 2B) Signature response as a function of reported intensity, for conditions rated as warm (lower left) and those rated as painful (upper right). Loess smoothing was used to visualize the relationship; shaded areas show bootstrapped S.E.M. The vertical line divides conditions explicitly rated as painful vs. non-painful, and the dashed horizontal line is the classification threshold that maximizes the decision accuracy for Painful vs. Non-painful (1.32; see Table 1). Pain/no-pain discrimination performance was evaluated graphically for comparisons reported in Table 1. Performance (points) was generally better than predicted by the Gaussian model (lines), suggesting a super-Gaussian distribution of signature response. Forced-choice discrimination showed 100% sensitivity/specificity in all comparisons.

FIG. 3 shows the application of the signature of FIG. 1A to physical and social pain stimuli, as evaluated in Study 3. FIG. 3A) Signature response by condition. The dashed horizontal line shows the threshold derived from Pain vs. Warm classification in Study 1. Error bars show SEM. ROC plots for the forced-choice discrimination, assessed only from the pattern within a single region of interest shown in the inset (FIG. 3B is anterior insula/operculum) (FIG. 3C is anterior cingulated cortex) (FIG. 3D is S2/Posterior insula). A physical pain signature would ideally show high sensitivity and specificity for Pain vs. Warm (squares) and Pain vs. Rejector (closed circles), but chance performance for Rejector vs. Friend (open circles). Insets: positive (light) and negative (dark) signature weights in each region of interest, with high- vs. low-magnitude weights in solid vs. transparent.

FIG. 4 shows the analysis of head movement in Study 1. Three translation (A, C) and three rotation (B, D) parameter estimates, based on image realignment, are plotted as a function of time within the heat trial (A, B) and stimulus temperature (C, D). In each case, the average absolute displacement from the previous image is plotted on the y-axis. Error bars show standard error of the mean. Head movement did not increase during stimulation or at stimulus onset and offset. Rather, a modest movement increase is observed at the onset of the pain-predictive cue. Movement was not significantly predicted by temperature for any movement direction.

FIG. 5 shows a schematic presentation of the preprocessing and analysis stages of the fMRI patterns. The preprocessing and first-level General Linear Model (GLM) are standard steps performed with SPM software, with the exception of outlier identification and percent-change scaling. Activity maps from the GLM are cross-multiplied by the signature map, which was developed using a separate cross-validated machine learning regression (not illustrated), to yield a scalar signature response value for each image. Signature response values are used to predict continuous pain and in classification.

FIG. 6 shows the development of the neurologic signature based on data from Study 1. A) A mask of a priori regions used in analysis based on the Neurosynth database, associated with ‘pain’ at q<0.05 FDR-corrected. In all plots, yellow indicates positive predictive weights for pain, and blue indicates negative weights. B) Unthresholded signature pattern weights from the LASSO-PCR analysis, shown as Z-scores, with voxels with lower Z-scores more transparent. The black outline shows the a priori mask boundaries. Blue/yellow indicate Z<−2 and Z>2, respectively. C) Map thresholded at q<0.05 FDR (P<0.003) for display. Blue/yellow indicate Z<−3 and Z>3, respectively. D) Histograms of prediction error and prediction-outcome correlation from nonparametric permutation test. Histograms show the distribution of null-hypothesis results, and the red line shows the actual solution.

FIG. 7 shows the correlation of the neurologic signature response with the time course of objective stimulus delivery vs. reported pain in Study 1. A) Signature response (scaled to reflect predicted temperature) across time within trials. Lines/shading: means/standard errors across participants. Pattern expression increased monotonically with temperature only following stimulation, and not during cue and pain report periods. B) Top: Time-course of thermal stimulation (light) and subjective pain (dark; shaded area: SEM). Bottom: Predicted fMRI activity, convolving the stimulus and report time-courses with SPM's standard double-gamma hemodynamic response function. The predictors were correlated (r=0.78, 61% of variance shared), but the pain time course peaked appreciably later. C) Correlation between the time course of signature temperature effects and the model were higher for the pain report model (dark) than the stimulation time course model (light) for every individual tested. Correlations for individual subjects are shown by points connected with light gray lines.

FIG. 8 shows the neurologic signature response to the analgesic remifentanil in Study 4. A) The signature from Study 1 applied to Painful (red) and Warm (blue) events across trials. The gray box marks the intravenous drug infusion period. Average model fits with SEM across individuals (shaded areas) are shown. The model captured the effects of drug effect site concentration and the infusion period itself on responses to Painful and Warm events; thus, the curves reflect a combination of potential drug and psychological effects across time. B) Average profile of drug effect site concentration based on the pharmacokinetic model of Minto et al (DaSilva A F, et al. J Neurosci 2002; 22:8183-92). The observed signature responses parallel the time course of effect site concentration and show no effect of Open vs. Hidden administration. Both findings suggest that signature responses are mainly influenced by the drug itself, rather than expectations about drug delivery.

### DESCRIPTION OF INVENTION

Described herein is a brain-based neurologic signature that serves as a biomarker of physical pain. As further described herein, the neurologic signature is indicative of pain, discriminates physical pain from other pain, is sensitive to the analgesic effects of opioids and can predict pain intensity at the level of the individual person. The neurologic signature can be applied to individuals in the diagnosis and treatment of pain related neuropathic conditions, as well as to compare efficacy of therapeutic treatments. Accordingly, further described herein are methods for detecting pain, diagnosing pain related conditions, and determining efficacy of an analgesic using the neurologic signature.

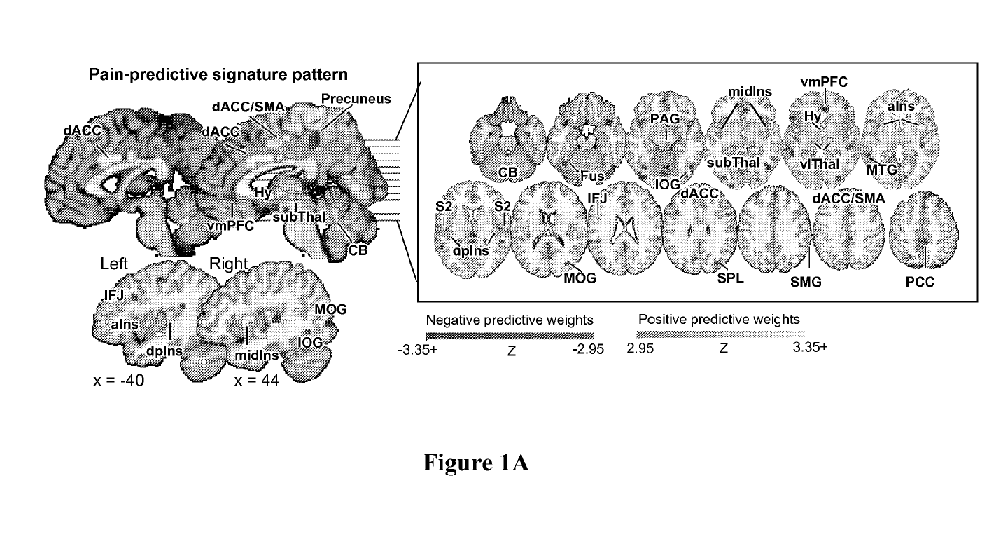

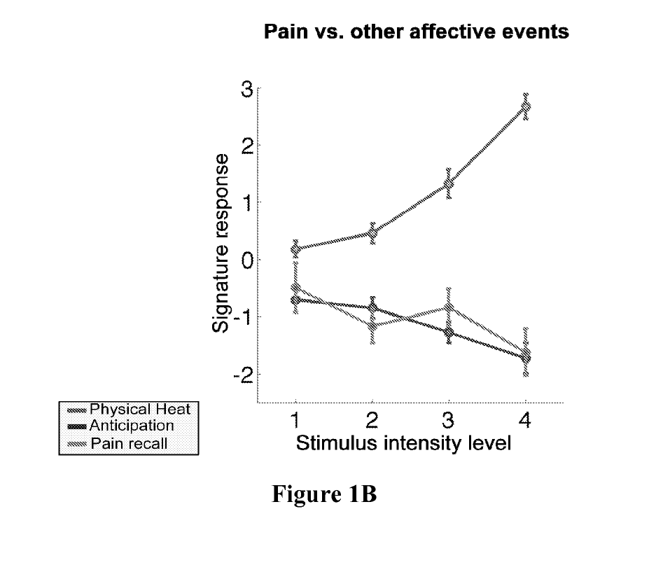

The neurologic signature (also referred as a signature map or normative map or reference map), comprises an fMRI pattern that is indicative of physical pain in a subject. In one embodiment, the neurologic signature comprises an fMRI pattern that is least about 60% identical to the fMRI pattern shown in FIG. 1. The identity may be in terms of overlapping brain voxels or shared variance. The term “voxel,” as used herein, refers to a point or three dimensional volume from which one or more measurements are made. A voxel may be a single measurement point, or may be part of a larger three dimensional grid array that covers a volume. In various embodiments, the neurologic signature comprises an fMRI pattern that is at least about 65%, or at least about 70%, or at least about 75%, or at least about 80%, or at least about 85%, or at least about 90%, or at least about 95% identical, or at least about 96% identical, or at least about 97% identical, or at least about 98% identical, or at least about 99% identical (or any percent identity between 60% and 99%, in whole integer increments), to the fMRI pattern of FIG. 1A. In one embodiment, the neurologic signature comprises an fMRI pattern that is substantially identical to the fMRI pattern shown in FIG. 1A. In one embodiment, the neurologic signature comprises the fMRI pattern shown in FIG. 1A.

The development and validation of the neurologic signature is described in detail in Examples 1-5. As described in the Example 2 (describing Study 1), machine-learning analyses identified a neurologic signature comprising a pattern of fMRI activity across brain regions, that was associated with heat-induced pain and could predict pain at the level of the individual person. The pattern included brain regions including thalamus, posterior/anterior insula, SII, anterior cingulate, periaqueductal gray, and other regions. The neurologic signature showed≧94% sensitivity and specificity in discriminating painful heat from non-painful warmth, pain anticipation, and pain recall (95% confidence interval [CI]: 89-100%). The signature discriminated painful heat from non-painful warmth with 93% sensitivity and specificity (CI: 84-100%) (Example 3 describing Study 2); and physical pain from social pain with 85% sensitivity (CI: 76-94%) and 73% specificity (CI: 61-84%), and 95% sensitivity/specificity in a forced-choice test (Example 4 describing Study 3). Furthermore, the signature's strength was substantially reduced by the analgesic remifentanil (Example 5 describing Study 4).

We used the signal values from the voxels, each of which measured 3 mm³, in the a priori map to predict continuous pain ratings, using leave-one-participant-out cross-validation. The result was a spatial pattern of regression weights across brain regions, which was prospectively applied to fMRI activity maps obtained from new participants. Application of the signature to an activity map (e.g., a map obtained during thermal stimulation) yielded a scalar response value, which constituted the predicted pain for that condition.

In another embodiment, the present invention includes a method of detecting pain in a subject using the neurologic signature of the present invention. The method comprises applying a stimulus to the subject and measuring the brain or neuronal activity in the subject in response to the stimulus by fMRI to generate a brain map of the subject.

It is noted that although the signature map was developed in response to an experimental thermal stimulus, it is believed that the map is applicable to pain induced by a variety of stimuli and is useful to predict pain in response to a variety of stimuli. Accordingly, the subject may be given any sensory stimulus to induce pain. Examples of stimuli include without limitation, thermal (heat or cold), mechanical (such as a touch or a pinprick), electrical, ischemic, tissue injury, or administration of a compound (chemical).

The brain map of the subject (or subject map) comprising an fMRI pattern induced in the subject in the response to the stimulus is then compared to the neurologic signature map of the present invention. In some embodiments, the term comparing comprises applying the neurologic signature to the brain activity map of the subject to produce a signature response value.

In some embodiments, the term comparing means evaluating the brain activity in a particular region or voxel of the subject map to the corresponding region or voxel in the signature map in order to identify similarities or dissimilarities between the fMRI patterns of the two maps.

In some embodiments, the connectivity values among brain regions specified in the subject map are compared with the connectivity values in the signature map. “Connectivity” is a known term in the field of human neuroimaging, and refers to the assessment of the strength or pattern of statistical relationships among regions. In some embodiments, it refers to the strength of relationships among regions specified in the brain map (or portions of it), as summarized by metrics such as Pearson's correlation coefficients among regions, nonparametric correlations such as Kendall's Tau, Kruskal's Gamma, Spearman's Rho, and similar metrics; graph theoretic measures including Centrality, Path Length, Small-worldness, and similar measures of global connectivity; or other measures of similarity or dissimilarity in functional relationships.

Connectivity may reflect functional connectivity, defined here as the relationship between activity measures in two or more regions over time assessed with fMRI, Positron Emission Tomography, Arterial Spin Labeling fMRI, or related methods; or structural connectivity, defined here as measures related to the integrity of white-matter (axonal) tracts connecting two or more regions defined by the neurologic signature pattern, as assessed using diffusion-weighted imaging, including diffusion-tensor imaging, diffusion-spectrum imaging, high angle resolution diffusion imaging, or similar techniques. The present invention includes methods comparing connectivity measures among brain regions defined by all or part of the neurologic signature pattern, either quantitatively by comparing samples from an individual person of interest to other normative connectivity samples, or by qualitative assessment (i.e., by a physician).

The comparison and analyses of the subject's fMRI data may be performed by a computer to provide an output. In some embodiments, such output may be a single numeric value or it may be a series of numeric values. The comparison and analyses of the fMRI data may also be performed by an individual, such as a physician. Analysis of fMRI data may be performed using standard statistical methods. Methods for statistical analyses of comparison of fMRI patterns are well known in the art and are incorporated herein. A number of computer programs based on pattern recognition or machine learning methods for the analysis of fMRI data are well known in the art and are commercially available (e.g. MATLAB Medical image Analysis) and may be used in methods of the present invention.

The analysis and determination of similarity and/or the dissimilarity between the signature map and the subject map yields information that may be used as the basis for diagnosis of pain-related conditions and treatments. For example, the subject map may comprise an fMRI pattern that is identical or substantially similar to the signature pattern indicating the presence of pain in the subject but may vary in terms of the intensity or the magnitude of the signature, providing a measure of quantification of pain in the subject. In some instances, the subject map may comprise an fMRI pattern that is dissimilar from the signature map in that the subject map may comprise a pattern that shows different levels of brain activity in different portions of the map as compared to the corresponding portions of the signature map. In some instances, the subject map may comprise a pattern that exhibits different relationships among the activity levels in one or more portions of the subject map, or “connectivity,” as compared to the corresponding portions in the signature map.

Thus, in one embodiment, the method comprises applying the signature map to the subject map to provide a scalar response value. The scalar response value is a numerical value that reflects the magnitude of the signature in the subject and provides a means of quantifying the pain. For example, a higher scalar response value would indicate a greater degree of pain in the subject and a lower scalar response value would indicate a lower degree of pain the subject. In some embodiments, the method further comprises quantifying the pain in the subject based on the response value.

In some embodiments, the method comprises diagnosing a pain related condition based on the comparison between the subject map and the signature map. Such conditions include without limitation, hyperalgesia, allodynia, pain catastrophizing, fear of pain, chronic neuropathic pain including complex regional pain syndrome or reflex sympathetic dystrophy, post-stroke pain, and other chronic widespread pain conditions, inflammatory pain, and nociceptive pain. For example, a high scalar response value to a standard pain stimulus may indicate presence of hyperalgesia or chronic pain in the subject. Similarly, a subject map that exhibits substantial similarity to the signature map in a response to an innocuous stimulus, such as a light touch, may indicate presence of allodynia in a subject. Dissimilarities between the two maps with respect to the brain activity in one or more portions of the subject map, or relationships among activity in one or more portions of the map, may indicate presence of complex regional pain syndrome or chronic pain. A number of brain regions have been implicated in pain and based on the knowledge in the art, one skilled in the art will be able to interpret the results of the comparison between the subject map and the signature map, or use quantitative metrics from normative populations to serve as distribution against which anomalous neurophysiological features related to chronic pain may be detected.

In some embodiments, the method further comprises administering a therapeutic treatment to the subject. The term therapeutic treatment means a regimen intended to have a preventive, ameliorative, curative, or stabilizing effect. Examples of therapeutic treatment include pharmaceutical analgesics, physical treatment (e.g., massage or acupuncture), electrical treatment, thermal treatment, electromagnetic radiation, counseling, or a surgical, medical, or dental procedure. The term “analgesics” includes any drug that is used to achieve relief from pain, and includes without limitation, organic compounds, inorganic compounds, peptides or proteins, and nucleic acids. In some embodiments, the therapeutic treatment comprises administration of an analgesic. The type and the dosage of the analgesic to be administered may be selected on the basis of the comparison of the subject map and the signature map.

In some embodiments, the method further comprises measuring another indicator of pain. Such indicators may be verbal or non-verbal. Non-verbal indicators may be vocal such as sighs, gasps, moans, groans, cries or non-vocal such as facial grimaces, winces, bracing, restlessness etc. In some subjects such indicators may be consistent with the level of pain detected by the brain map and provide verification of the level of pain predicted by the claimed method. In some subjects such indicators may be inconsistent with the level of pain detected by the brain map and may indicate the presence of a neuropathic pain-related condition such as hyperalgesia or allodynia, or the presence of pain with an emotional rather than nociceptive basis, or the presence of pain with a non-normative neurophysiological basis.

In another embodiment, the present invention includes a method to diagnose a pain related condition in a subject comprising measuring brain activity by fMRI in a subject to generate a brain map of the subject and comparing the brain map of the subject to the signature map of the present invention to identify any dissimilarities between the structural and functional connectivity of the brain regions of the subject. In this embodiment, the subject's data reflects brain activity of the subject in the resting state or any other state whose purpose of assessment is to quantify structural or functional connectivity among brain regions. ‘Connectivity’ is an established general method in the field of human neuroimaging, and refers to the assessment of the strength or pattern of statistical relationships among regions. Here, it refers to the strength of relationships among regions specified in the neurologic signature map or part of the map, as summarized by metrics such as Pearson's correlation coefficients among regions, nonparametric correlations such as Kendall's Tau, Kruskal's Gamma, Spearman's Rho, and similar metrics; graph theoretic measures including Centrality, Path Length, Small-worldness, and similar measures of global connectivity; or other measures of similarity or dissimilarity in functional relationships.

Connectivity may reflect functional connectivity, defined here as the relationship between activity measures in two or more regions over time assessed with fMRI, Positron Emission Tomography, Arterial Spin Labeling fMRI, or related methods; or structural connectivity, defined here as measures related to the integrity of white-matter (axonal) tracts connecting two or more regions defined by the neurologic signature pattern, as assessed using diffusion-weighted imaging, including diffusion-tensor imaging, diffusion-spectrum imaging, high angle resolution diffusion imaging, or similar techniques. The present invention applies to methods comparing connectivity measures among regions defined by all or part of the neurologic signature pattern, either quantitatively by comparing samples from an individual person of interest to other normative connectivity samples, or by qualitative assessment (i.e., by a physician).

In another embodiment, the present invention includes a method for determining efficacy of a therapeutic treatment. The method comprises administering a therapeutic treatment to a subject, applying a stimulus to the subject and measuring brain activity of the subject in response to the stimulus to generate a brain map of the subject. The stimulus may be provided before, after or simultaneously with the administration of the treatment. The method further comprises comparing the brain map of the subject with the signature map of the present invention to identify similarities or dissimilarities between the two as discussed above. For example, a lower scalar response value upon administration of the treatment would be indicative of the efficacy of the treatment. The subject map may be further compared with a control subject map obtained from the same subject or another subject treated with placebo or treated with a therapeutic treatment with known efficacy.

One embodiment provides a requesting person or agency (e.g. an insurance company) with an objective numerical comparison of a patient with pain to pain-free persons. The results are based on the pattern and/or the percentage of neuron activation compared to standard pain-free persons when a pain-producing stimulus is applied at or near the suspected pain generator. Alterations and pattern changes will occur in pain processing between normal and pain subjects when the same stimulation is applied (such as heat, pressure, vibration or cold to the same anatomic area). This benefits insurance companies, courts, etc. as well as the pain patients themselves. Insurance company studies have shown an estimated 20% to 46% of litigation involving chronic pain and suffering is based on either fraudulent behavior or misrepresentations by the plaintiff Other insurance company-funded studies have shown that up to approximately 40% of the population feels that it is acceptable to misrepresent their pain and suffering symptomatology in order to obtain a favorable insurance or other settlement.

This embodiment also benefits the individual with a considerable pain who was not diagnosed as having pain when evaluated/examined in accordance with past practice. Without objective findings, a pain sufferer will occasionally go without appropriate compensation and/or further medical treatment, even though he/she will have continued pain and significant functional activity restrictions limiting his/her income, decreasing the quality of life, and/or impacting his/her family's future. The present fMRI signature can identify patients with significant pain, sort out the embellishers and fraudulent claims, and facilitate proper decision making for the appropriate institution or person.

As discussed above, the pain pattern and neuron activation in the brain of a patient with pain is different from that of persons with no such pain. Pain patients have an increased pain sensitivity, hyperalgesia and frequently also a central augmentation of pain. For example, a patient with lower back pain who receives a painful stimulus applied to his/her thumbnail will have an fMRI that differs from that for the control group when the same pain stimulus is applied. Differences in the brain regions and pattern of neuron activation between the two sets of fMRIs can be objectively observed. The chronic lower back pain patient will exhibit extensive common patterns of neuron activation of pain in related cortical areas.

Conversely, the intensity needed to observe a common pain level on the fMRI will be less for the chronic pain patient than for the pain-free persons. In addition, the chronic pain patient will normally have a different regional cerebral blood flow as compared to the pain-free control group.

The actual evaluation whether a given person claiming to suffer pain in fact has pain is conducted in an fMRI machine by initially placing the patient in a comfortable position within the bore of the magnet of the machine. The patient's head is immobilized, for example with a vacuum bean bag, a foam headrest and a removable plastic bar across the bridge of the nose, although if there is concern about a tremor or movement, a bite bar can be used instead to hold the head steady, and a pain stimulus is applied while the patient's brain is scanned at and an fMRI image of the brain activity is taken. To avoid the effect of sensitization, the pain stimulus is applied in a random order. The modality of the stimulus will also be random.

Members of the control group were previously subjected to the same pain stimulus at intervals, initially up to a sensation threshold level which lies just below the pain threshold level, and thereafter to the pain threshold level and, finally, to the maximum tolerable pain level, while their brains are scanned and fMRI images thereof are taken. The fMRI images of the members of the control group are statistically combined into a standard fMRI image or chart of the average brain activities of the members of the group. The standard chart is then stored, for example in a computer memory or other suitable memory or storage device.

The same protocol used for the control group is used on the pain patient by preferably applying the pain stimulus to the painful body part and the contralateral body part. It should be noted, however, that for purposes of the present invention the pain stimulus can be applied to parts of the body not affected with chronic pain in order to generate fMRI images that reflect the presence or absence of chronic pain.

This method of the present invention for processing claims by an asserted pain sufferer for reimbursement from an insurance company or any other third party involves initially receiving the request for compensation, for example at an insurance company. The request is referred to an evaluator who then examines the patient by applying pain stimuli to the patient in the manner described above. With the pain stimulus applied, an fMRI image of the patient's brain activity is prepared. The patient's fMRI is then compared to the standard fMRI image or chart from the members of the control group or the fMRI signature of FIG. 1A, either by a computer (which compares the patient's fMRI with the standard fMRI and provides an output that reflects the difference between the two) or, in the alternative, by the evaluator, preferably but not necessarily a physician. The evaluator judges if the difference between the patient's fMRI and the standard fMRI is statistically significant, which means that the differences between the two fMRIs are sufficiently large so that they are not the result of random variations, but are caused by the presence of chronic pain in the patient. If the difference is judged to be statistically significant, the evaluator informs the requestor that the patient suffers chronic pain. Conversely, if the difference between the two images is judged to be statistically not significant, the evaluator informs the requestor (e.g. the insurance company) that the patient does not have chronic pain.

Although it is entirely feasible to leave the judgment whether the difference between the two sets of fMRIs is statistically significant to a computer analysis and use the output (e.g. a numerical output that is reflective of the difference) as the criterion whether the patient suffers chronic pain, for example whenever the difference rises above a predetermined threshold level, review of the respective images by a trained person, such as a physician, will typically be desirable, and he/she may supplement the computer output with additional comments concerning the computer output and/or the testing of the patient and the observed results.

The present invention also relates to systems that may be used in combination with performing the various methods according to the present invention. These systems may include a brain activity measurement apparatus, such as a magnetic resonance imaging scanner, one or more processors and software according to the present invention. These systems may also include means to present information to a device operator during testing, or upon completion of testing, or at a later time. These systems may also include software for automated diagnosis of the subject, or testing of brain activation metrics. These systems may also include mechanisms for communicating information such as instructions, stimulus information, physiological measurement related information, and/or subject performance related information to the subject or an operator. Such communication mechanisms may include a display, preferably a display adapted to be viewable by the subject while brain activity measurements are being taken. The communication mechanisms may also include mechanisms for delivering audio, tactile, temperature, or proprioceptive information to the subject. In some instances, the systems further include a mechanism by which the subject may input information to the system, preferably while brain activity measurements are being taken.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention.

The present invention also relates to software that is designed to perform one or more operations employed in combination with the methods of the present invention. The various operations that are or may be performed by software will be understood by one of ordinary skill, in view of the teaching provided herein.

In another embodiment, computer assisted method is provided comprising: measuring activity of one or more internal voxels of a brain; employing computer executable logic that takes the measured brain activity and determines an estimate of a condition of the subject computed from the measured activity; and communicating information based on the determinations to the subject or device operator.

### EXAMPLES

### Example 1

This example illustrates the methods of data acquisition and analysis used in the studies presented in Examples 2-5.

### Participants

All participants provided written informed consent. Studies were individually approved by the Columbia University Institutional Review Board. For all four studies, preliminary eligibility was assessed with a general health questionnaire, a pain safety screening form, and an fMRI safety screening form. Participants reported no history of psychiatric, neurological, or pain disorders. Ethnicity was assessed using self-report screening instruments prior to study procedures.

### Thermal Stimulation and Pain Rating

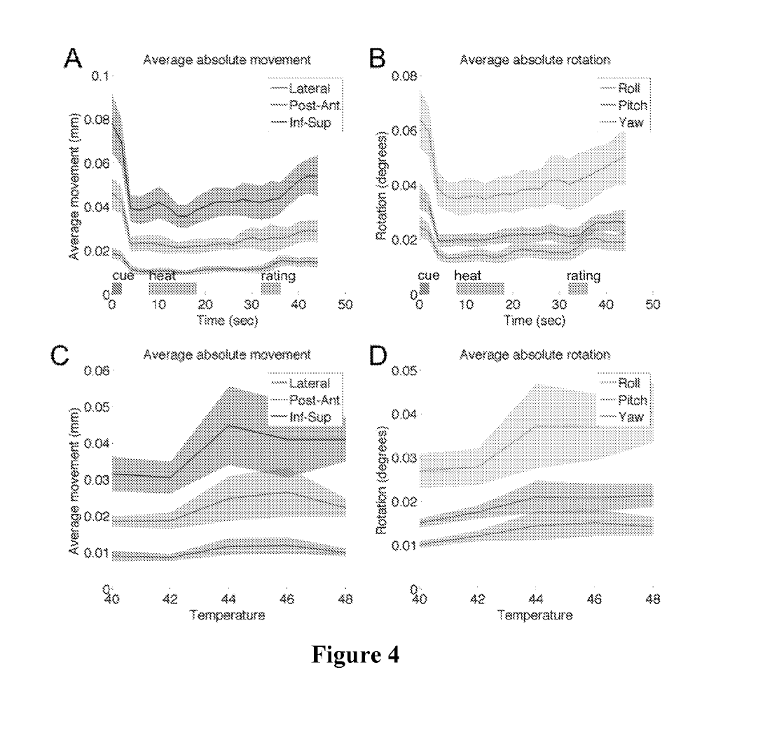

In all four studies, thermal stimulation was delivered to the volar surface of the left (non-dominant) inner forearm applied using a TSA-II Neurosensory Analyzer (Medoc Ltd., Chapel Hill, N.C.) with a 16 mm Peltier thermode end-plate. Each stimulus lasted 8-12 seconds, depending on the Study, and always included a period of time during which the stimulus ramped up from baseline temperature (32° C.) to the target temperature, and another steady ramp to baseline. The ramping was intended to help prevent head movement, and analyses described below confirmed that head movement does not increase at pain onset or during pain, and does not increase with increasing temperature (FIG. 4).

Before testing in Studies 1, 3, and 4, we performed a pain calibration procedure using methods described in previous work (Atlas L Y, et al. J Neurosci 2010; 30:12964-77; Buhle J, Wager T D. Pain 2010). In brief, we tested different sites on the forearm during calibration and used an adaptive staircase procedure to identify sites on the forearm with similar nociceptive profiles and to derive the individual participant's dose-response curve for the relationship between applied thermal stimulation and reported pain (slope, intercept, R²). In Study 2, all participants received the same temperatures.

General fMRI Processing

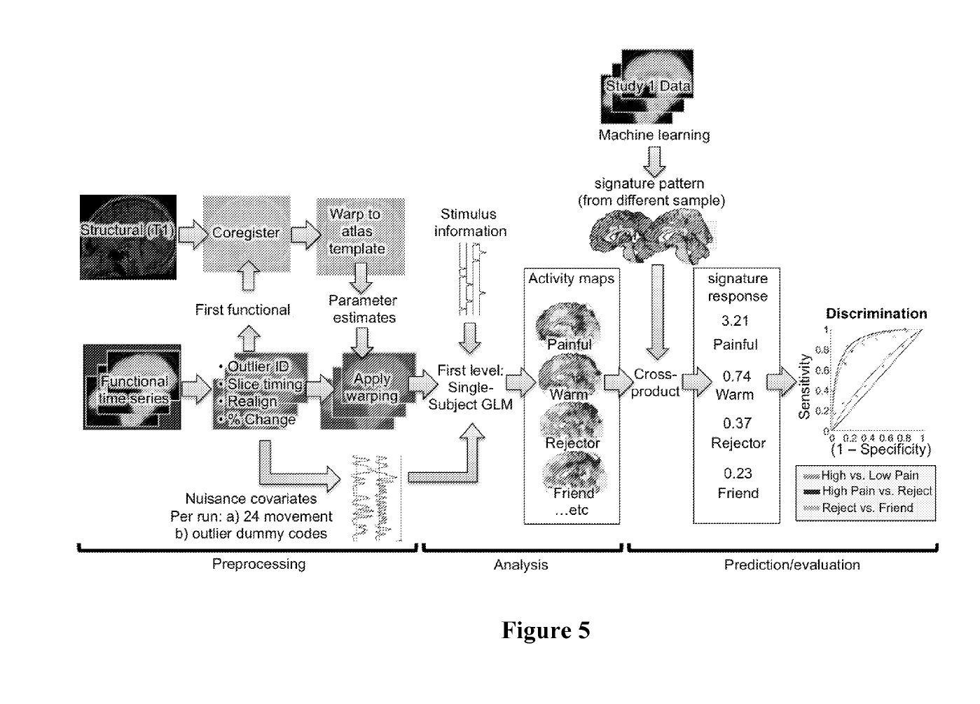

FMRI data for all three studies were subjected to a standard series of preprocessing and analysis steps, which are shown in FIG. 5. The stages consisted of Preprocessing, Analysis, and Prediction/Evaluation. Preprocessing included a sequence of commonly used procedures performed using SPM software (Wellcome Trust Centre for Neuroimaging, London, UK). SPM5 was used for Studies 1, 3, and 4. SPM8 was used for Study 2, but the algorithms for all the steps used were identical in both versions. Preprocessing also included several quality control procedures not typically performed in SPM per se, which were designed to be simple to implement (code can be obtained from wagerlab.colorado.edu or from the authors). Analysis consisted of a standard General Linear Model (GLM) analysis of each individual participant's data, and was conducted to summarize activity maps for painful heat and other conditions. Prediction involved estimating the signature response by computing the cross-product of these individual subject activation maps with a machine-learning signature pattern derived from other individuals. Specifically, the signature was derived from cross-validated machine learning analyses in Study 1 (see Signature Development below). It was applied to out-of-training-sample individual activity maps in Study 1 and new individual activity maps in Studies 2 and 3 to generate signature response values for each condition within each individual, which reflect a quantitative match to the pain signature pattern. Finally, evaluation involved quantifying the sensitivity and specificity of signature response to physical pain, and assessing the magnitude and significance of the opiate effect in Study 4.

These steps were employed for all analyses for all studies, except as noted below. Specifically, the initial Signature Development analyses involved several minor differences intended to ensure minimal artifacts in the data and minimize assumptions about the shape of the hemodynamic response to pain.

### Preprocessing

Structural T1-weighted images were subjected to the following steps (FIG. 5): Coregistration (SPM). We used SPM's iterative mutual information-based algorithm to coregister volumes to the mean functional image for each subject. Coregistration was manually checked by a trained analyst, and the starting point was adjusted and the algorithm re-run until the coregistration was satisfactory.

Warping to normative atlas (SPM). Structural images were normalized to MNI space using the generative Segmentation/Warping algorithm (Ashburner J, Friston K J. NeuroImage 2005; 26:839-51) using the default parameters (7×8×7 nonlinear basis functions) and resliced to standard 2×2×2 mm voxels. Data were resampled to 3×3×3 mm voxels before signature development analyses (to facilitate efficient storage and processing) and before calculating signature response in all studies.

Functional images were subjected to the following steps (FIG. 5): Outlier/gradient artifact detection (custom code). The purpose of this was to remove intermittent gradient and severe motion-related artifacts that are present to some degree in all fMRI data. On each individual scanning run, we identified image-wise outliers by computing both the mean and the standard deviation (across voxels) of values for each image for all slices. Mahalanobis distances for the matrix of slice-wise mean and standard deviation values (concatenated)×functional volumes (time) were computed, and any values with a significant chi-squared value (corrected for multiple comparisons based on the more stringent of either false discovery rate or Bonferroni methods) were considered outliers (less than 1% of images were outliers). For each voxel, outlier time points were imputed with the voxel's overall run mean. Next, data across the entire run were Windsorized to three standard deviations. This procedure is similar to those commonly employed by many groups (nitrc.org/projects/art_repair/). Slice-acquisition-timing correction (SPM) interpolates the data to correct for differences in the acquisition time for each slice. Image realignment (SPM) is a rigid-body (6-parameter) registration to the mean functional image, and helps correct for head movement during scanning Percent signal change conversion (custom code). Time series data for each voxel were converted to percent signal change based on a spatially smoothed baseline time series (16 mm FWHM). Warping to normative atlas (SPM). Warping parameters estimated from coregistered, high-resolution structural images were applied, and functional images were interpolated to 2×2×2 mm voxels.

### Analysis

Except for machine learning analyses (see Signature Development below), activity maps for each condition within each participant were estimated using the GLM. For each individual, a set of regressors was constructed for conditions of interest (e.g., heat at a particular temperature, aversive image presentation, etc.) using a stimulation epoch that lasted the duration of the event convolved with the canonical hemodynamic response implemented in SPM. The parameter estimates (regression slopes) for each condition thus provided an estimate at each voxel of the activation intensity for that condition. We also included a set of nuisance covariates designed to capture noise. These included, for each run: a) a constant term (intercept) for that run; b) dummy regressors for estimated outlier images from preprocessing, which varied in number depending on how many outliers were detected but was nearly always<1% of images; and c) 24 movement-related covariates based on estimated movement during realignment, including 6 mean-centered motion parameter estimates, their squared values, their successive differences, and squared successive differences. Previous work has shown this to be helpful in reducing noise variance, violations of normality, and autocorrelation (Lund T E, et al. NeuroImage 2006; 29:54-66).

### Prediction and Evaluation

All assessments of performance were made at the level of the individual subjects, always based on a signature developed in other individuals using cross validation (Study 1) or simply applying the signature developed in Study 1 to new studies (Studies 2 and 3). For all tests, the signature response (BR) was estimated for each test subject in each test condition by taking the dot product of vectorized activation images (\vec{β}ₘₐₚ) with the signature pattern \vec{w}ₘₐₚ, i.e., (BR=\vec{β}ₘₐₚ^{T}\vec{w}ₘₐₚ), yielding a continuous scalar value. This value depends on the voxel size, but can be scaled based on the voxel volume. Values reported in this paper are for 27 mm³ voxels (i.e., 3×3×3 voxels). BR values derived from maps resliced to 2×2×2 mm voxels can be put on the same scale by multiplying by 27/8. We summarized the performance of the signature response in two ways: First, we assessed average prediction error (PE, the mean absolute deviation of predicted from observed pain ratings) when predicting continuous pain ratings. Second, we calculated sensitivity, specificity, positive predictive value, and effect sizes related to binary classification. We assessed binary classification decisions for painful stimulation relative to non-painful warmth, pain anticipation, pain recall, and social pain-inducing events.

We performed two kinds of binary classification tests. In the pain/no-pain test, sensitivity is the probability of a positive test—i.e., that the signature response was above a given criterion threshold—given that a person experienced pain (vs. one of the comparison conditions below). Specificity is the probability of a negative test given that a person experienced a condition other than pain. Positive predictive value is the probability that pain (vs. a comparison condition) was experienced given a positive test result. Effect size provides a continuous measure of the ability of the signature to separate pain from a comparison condition, and is reported as both (1) dₐ, a measure of the distance between the mean signature response in the pain-present vs. pain-absent conditions, divided by their pooled standard deviation, and (2) the area under the Receiver Operating Characteristic (ROC) curve (AUC), estimated directly using numerical integration of the ROC under all threshold values that yielded unique sensitivity/specificity values (0.5 is chance, and 1 is perfect discrimination). In the forced-choice discrimination test, signature response is compared for two conditions tested within the same individual, and the higher is chosen as more painful. In the forced-choice test, the ROC curves are symmetrical, and sensitivity, specificity, and positive predictive value are equivalent to each other and to decision accuracy (i.e., the probability with which the more painful of the two conditions is selected).

The forced-choice test has several advantages that make it particularly useful in the fMRI setting. First, the forced-choice test is ‘threshold free’ in the sense that an absolute decision threshold across individuals is not required; zero is used as the threshold for the difference between the two paired alternatives. Thus, individual differences in the shape and amplitude of the blood oxygen level dependent (BOLD) fMRI response (Handwerker D A, et al. NeuroImage 2012; Aguirre G, et al. NeuroImage 1998; 8:360-9) do not add noise in this kind of test. In addition, as the amplitude of the BOLD response varies as a function of field strength and scanner noise, the threshold in the pain/no-pain test must be calibrated for different scanners and field strengths (see, e.g., the thresholds for Study 1, collected at 1.5 T, vs. Study 2, collected at 3.0 T, in Table 1). Second, the forced-choice test likely provides a more realistic assessment of the signature's performance for validation purposes. Prediction error and sensitivity/specificity in the tests is calculated assuming that pain reports always accurately reflect experienced pain intensity in the normative samples we test here (i.e., a person reporting a “5” on the visual analogue scale always experiences more pain than a person reporting a “4”). However, this may not always be the case. Individuals may use the rating scale in somewhat different ways (e.g., the same experience may be reported by one person as a “5” on the visual analogue scale and by another as a “4”), which can reduce the apparent performance of even a perfect diagnostic test. Forced-choice discrimination performance does not require this assumption, as two conditions are compared within the same individual. The only condition that must hold for the ‘ground truth’ to be accurate is that an individual's pain reports must increase monotonically with pain experience; more pain should be reported as more painful.

TABLE 1

| Classification | | | | | | | | |
|---|---|---|---|---|---|---|---|---|
| performance | Pain/no-pain discrimination | | | Effect size | | | Binomial Forced-choice | |
| across studies | Threshold | Sensitivity | Specificity | PPV | AUC | dₐ | P-value | Sens./Spec./PPV^{f} |
| Study 1 | | | | | | | | |
| Painful vs. Warm^{a} | 1.40 | 95% (86-100%) | 95% (86-100%) | 95% (85-100%) | 0.95 | 2.69 | P < .001 | 100% (100-100%) |
| Pain vs. Anticipation | 0.36 | 100% (100-100%) | 99% (96-100%) | 95% (86-100%) | 0.99 | 3.69 | P < .001 | 100% (100-100%) |
| Pain vs. Pain Recall | 0.54 | 95% (85-100%) | 94% (89-98%) | 79% (64-92%) | 0.96 | 2.35 | P < .001 | 100% (100-100%) |
| Study 2 | | | | | | | | |
| Painful vs. Warm^{b,c} | 1.32 | 93% (84-100%) | 93% (84-100%) | 93% (84-100%) | 0.92 | 1.54 | P < .001 | 100% (100-100%) |
| Painful vs. near-thresh^{e} | 2.50 | 88% (77-97%) | 85% (72-95%) | 85% (73-96%) | 0.88 | 1.74 | P < .001 | 100% (100-100%) |
| High vs. low warmth | 1.00 | 56% (36-75%) | 100% (100-100%) | 100% (100-100%) | 0.79 | 1.31 | P < .01 | 100% (100-100%) |
| Study 3 | | | | | | | | |
| Painful vs. Warm | 1.40^{d} | 85% (76-94%) | 78% (67-89%) | 80% (68-89%) | 0.86 | 1.64 | P < .001 | 93% (86-98%) |
| Painful vs. Rejector | 1.40^{d} | 85% (76-94%) | 73% (61-84%) | 76% (65-86%) | 0.88 | 1.83 | P < .001 | 95% (89-100%) |
| Photo | | | | | | | | |
| Rejector vs. Friend Photo | 1.40^{d} | 27% (16-38%) | 88% (79-95%) | 69% (50-88%) | 0.57 | 0.31 | P = 0.22 | 56% (43-69%) |
| Study 4 | | | | | | | | |
| Hot vs. Warm, pre-drug | 1.40^{d} | 90% (79-100%) | 81% (65-95%) | 83% (67-95%) | 0.89 | 1.61 | P < .001 | 90% (79-100%) |
| Hot vs. Warm, on-drug | 1.61 | 86% (73-96%) | 62% (42-80%) | 69% (52-84%) | 0.74 | 1.01 | P < .01 | 76% (61-90%) |
| Hot pre-drug vs. on-drug | 1.61 | 86% (72-96%) | 62% (43-79%) | 69% (54-83%) | 0.74 | 1.01 | P < .01 | 76% (60-92%) |

^{a}Painful conditions were defined as those >44.5° C. and >5.80 average VAS units, and Warm as conditions <44.5° C. and <3.34 VAS units.
^{b}Study 2 was conducted on a scanner with a different field strength (3T), so the threshold was re-estimated.
^{c}Participants made painful vs. non-painful judgments on each trial.
^{d}The threshold derived from Study 1 was applied.
^{e}Participants made continuous, 100-point VAS ratings for pain or warmth intensity (0-99 for warmth, 100-200 for pain). Painful: >125, near-threshold: 75-125, high-warmth: 50-100, low-warmth: 0-50.
^{f}For two-choice (forced-choice) discrimination, the decision threshold for the difference between paired observations is 0. The sensitivity, specificity, and positive predictive value (PPV) are the same, and are equal to the decision accuracy.
AUC: Area under the Receiver Operating Characteristic curve; chance is 0.5.
PPV: Positive predictive value. da: Discriminability, a measure of effect size under a Gaussian model. Performance varies across studies based on the number of trials averaged to form condition maps.
Study 1: 12 trials each in Painful and Warm conditions.
Study 2 averaged 24 ± 13 trials (S.D.) for Pain, and 36 ± 9 trials for Warm, depending on ratings.
Study 3: 8 trials each in Painful and Warm conditions.
Study 4: 3 trials in each cell of the Hot vs. Warm x Pre- vs. On-drug design.

### Example 2

This example illustrates Study 1, which shows the development of the neurologic signature.

### Participants:

Study 1 included 20 participants (aged 28.8±7.5 [S.D.] years, 8 females). The sample consisted of 79% Caucasian, 5% Hispanic, and 16% African American participants. Data were collected between 2005-2006.

### Materials and Procedures:

fMRI Task Design

fMRI images were acquired during 8 functional runs (8 trials/run, 64 trials). The thermode was placed on a different skin site for each run, with two total runs per skin site, and 12 trials at each of 4 target pain intensities—non-painful warmth (Level 1), low pain (Level 3), medium pain (Level 5), and high pain (Level 7)—were delivered across the runs. Temperatures were selected for each individual based on a thermal pain calibration procedure (see above, “Thermal stimulation and pain ratings”). At the start of each trial, a square appeared in the center of the screen for 50 ms, followed by the presentation of a cue. The cue consisted of a male or female face showing a happy or fearful expression (33 ms) followed by a mask consisting of the same face presented for 1467 ms. Participants were not aware of the type of emotional face presented, and all analyses collapse across the different face types to examine brain activity as a function of temperature and reported pain.

During each trial, cues (2 sec) were followed by a six-second anticipatory interval during which a fixation cross was presented on the screen. Then, thermal stimulation was delivered at one of the four intensities, followed by a 14 sec rest interval during which participants fixated on a cross. The words “How painful?” then appeared on the screen for four seconds above a 9-point visual analogue scale (VAS), and participants rated the intensity of the stimulus using an fMRI-compatible track-ball (Resonance Technologies, Inc.) Continuous responses were recorded, with resolution equivalent to the screen resolution (approximately 600 discrete values).

fMRI Acquisition and Analysis

Image Acquisition.

Whole-brain fMRI data were acquired on a 1.5 T GE Signa Twin Speed Excite HD scanner (GE Medical Systems) at Columbia University's Program for Imaging in Cognitive Science (PICS). Structural images were acquired using high-resolution T1 spoiled gradient recall images (SPGR) for anatomical localization and warping to a standard space. Functional images were acquired with an echo-planar imaging sequence (EPI; TR=2000 ms, TE=34 ms, field of view=224 mm, 64×64 matrix, 3.5×3.5×4.0 mm voxels, 29 slices), and were resliced to 3×3×3 mm voxels after inter-subject normalization. Each run lasted 6 minutes and 18 seconds (189 TRs). Stimulus presentation and behavioral data acquisition were controlled using E-Prime software (PST Inc.).

Preprocessing.

Preprocessing was identical to that described in the General Methods, except that a) an additional denoising step was used to minimize artifacts for signature development, and b) FSL software was used for realignment. Denoising used a component-based strategy similar to published work (Thomas C G, et al. NeuroImage 2002; 17:1521-37; Tohka J, et al. NeuroImage 2008; 39:1227-45). We estimated the first 10 principal components (PCs) on the images from each scanning run, before any other processing. We constructed a task-related design matrix with the trail onsets convolved with the canonical HRF (no temperature information was entered to avoid bias), and a nuisance-related design matrix based on head movement parameters and outlier time points identified as described above. Components that appeared clearly artifactual (e.g., those expressed only at the edge of the brain, those that included an obvious single spike, etc.) and were related to the nuisance regressors but not the task, were removed (1.06±0.59 (S.D.)

Signature Development Analysis.

Signature development analyses were conducted on Study 1 using custom Matlab code (Wager T D, et al. Science 2004; 303:1162-7) implementing LASSO-PCR, a cross-validated, regularized regression procedure. LASSO, or Least Absolute Shrinkage and Selection Operator-regularized regression (Tibshirani R. Journal of the Royal Statistical Society, Series B 1996; 58:267-88), was implemented in Matlab by Guilherme Rocha and Peng Zhao. This was embedded within a leave-one-subject out cross-validation loop that first used principal components-based data reduction so that selection was performed on components, as described in previous work (Wager T D, et al. J Neurosci 2011; 31:439-52). The resulting pattern of regression weights constituted the signature, which was applied to average pain maps and general linear model-based activation maps in Studies 1-3. All predictions made for Study 1 data were cross-validated (see below).

The signature development analysis consisted of five steps: 1) Feature selection: Voxels within an a priori mask of pain-related brain regions was selected based on prior literature; 2) Data averaging: Data during pain from each in-mask voxel were averaged within each stimulus intensity for each individual, to generate 4 pain-related activation maps per individual; 3) Machine learning: LASSO-PCR was run using those maps to predict pain reports; 4) Bootstrapping was used provide P-values for voxel weights in order to threshold the signature weights for display and interpretation; and 5) Permutation tests were used to validate the unbiased nature of the procedure.

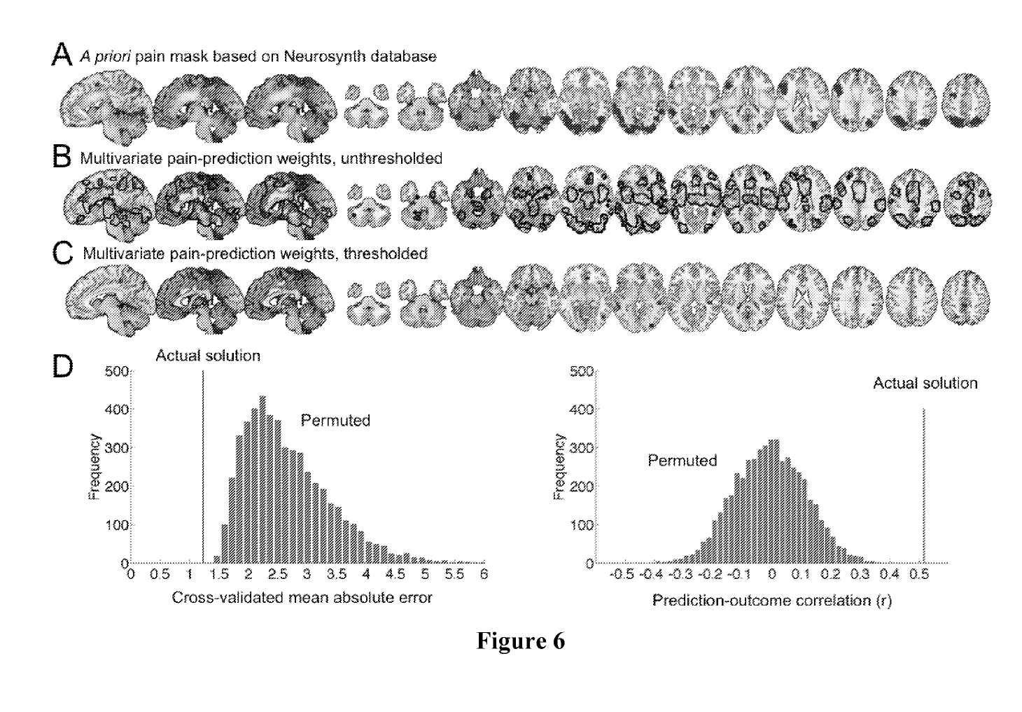

Feature selection. To accomplish Step 1, the automated meta-analysis toolbox Neurosynth (neurosynth.org) was used to a create a mask based on a meta-analysis of previous studies that frequently use the word ‘pain’ to select voxels a priori (Yarkoni T, et al. Nature Methods 2011). The mask (see FIG. 6A, top) was based on regions showing consistent results across 224 published studies (out of 4,393 total studies in the database) in a ‘reverse inference’ analysis, which was a chi-squared analysis of the 2×2 contingency table of counts of [activated (within 10 mm) vs. non-activated]×[pain vs. non-pain] within each voxel. Studies were counted as involving ‘pain’ if they mentioned ‘pain’ more than 1 time per 1000 words in the study (the default value in neurosynth) and thresholded at q<0.05 False Discovery Rate (P<0.0072) corrected. The mask included 22,379 positive voxels (2×2×2 mm, resliced to 3×3×3 mm for analysis) in which activity positively predicted pain (6.35% of the volume of the standard SPM5/8 brain mask brainmask.nii) and 10,940 negative voxels in which activity negatively predicted pain (3.1%), for a total of 9.45% of the in-brain volume. Weights from all voxels in this mask were used to estimate signature response and make predictions (no further thresholding was used for predictive purposes).

Data averaging. To accomplish Step 2, we averaged data within each trial in each voxel over the period 8-24 seconds after heat onset, and then averaged across the 12 trials for each stimulus intensity. This time window was chosen a priori based on the approximate time when reported pain is high from previous work (Baliki M N, et al. J Neurophysiol 2009; 101:875-87; Lindquist M A, et al. NeuroImage 2009; 45:S187-S98; Wager T D, et al. Science 2004; 303:1162-7; Bornhovd K, et al. Brain 2002; 125:1326-36; Koyama Y, et al. Pain 2004; 107:256-66) which is later than typical responses for a similar stimulation epoch due to temporal summation and hemodynamic lag in pain-related activity. Simple averaging has the advantage of simplicity and lack of strong assumptions about the shape of the hemodynamic response, although improvements in the use of timing information is a rich direction for future improvement that has already started to be explored (Grosenick L, et al. IEEE Trans Neural Syst Rehabil Eng 2008; 16:539-48).

Machine learning. To accomplish Step 3, we used cross-validated LASSO-PCR with activation maps from each condition within participants as the predictor, and average pain reports from each condition within participants as the outcome. The linear algorithm provided interpretable brain maps composed of linear weights on voxels, which is a substantial advantage over nonlinear kernel methods. We did not explore nonlinear methods.

We used leave-one-subject-out cross-validation to estimate prediction error (PE; mean absolute deviations between predicted and actual temperatures) on new trials. This standard approach in machine learning involves dividing the sample into a training set (all but one participant) and a test set (the test participant). LASSO-PCR was used to estimate regression weights for each voxel from the training dataset (\vec{w}ₘₐₚ, the signature pattern), and then predictions were made for the test participant by taking the dot-product of the test brain activation maps (\vec{β}ₘₐₚ) and the signature pattern (\vec{β}ₘₐₚ•\vec{w}ₘₐₚ). This yielded a scalar predicted pain value (the signature response) for each condition, and prediction error was quantified. The procedure was repeated 20 times (once for each participant) so that each trial was part of the test set exactly once. This procedure yields minimally biased estimates of prediction accuracy for new participants (there is a slight bias in accuracy towards zero, as with all cross-validation methods). Weight maps applied to Study 1 were always based on data from out-of-test-sample individuals, and the final signature weights (applied to Studies 2-4) were based on the full Study 1 sample.

To apply the signature to new activation maps across multiple conditions (i.e., anticipation, stimulation, and pain recall at each intensity, and other maps in Studies 2-4), we used a standard general linear model (GLM) with the canonical SPM hemodynamic response function to simultaneously estimate activation maps (\vec{β}ₘₐₚ) for each condition, and then applied the signature pattern (\vec{β}ₘₐₚ•\vec{w}ₘₐₚ) to yield a scalar signature response value for each condition. The signature response values are thus predictions of the magnitude of pain for a given condition, and their values across conditions can be compared and tested.

In our initial analyses of Study 1, we compared LASSO-PCR results with those from another popular method, Support Vector Regression (SVR; Smola A J, Schölkopf B. Statistics and computing 2004; 14:199-222) in order to check whether predictions were similar and whether SVR produced similar accuracy levels. Predictions and accuracy levels were nearly identical with SVR in all cases (predictions between LASSO-PCR and SVR were correlated>r=0.99 in most cases), so we do not focus on the SVR results. We prefer the LASSO-PCR results for transparency and consistency with our previous work. LASSO-PCR and SVR produced very similar results in all analyses we performed, and we do not consider the choice of algorithm to be critical, though algorithms that yield improved results could be developed.

Bootstrap tests. To accomplish Step 4 and threshold voxel weights for interpretation and display, we constructed 5,000 bootstrap samples (with replacement) consisting of paired brain and outcome data and ran LASSO-PCR on each. Two-tailed, uncorrected P-values were calculated for each voxel based on the proportion of weights below or above zero, as in previous work (1, 20), and subjected to False Discovery Rate correction (P<0.0028, 355 significant voxels; FIG. 6B, C). The signature weight map applied to Studies 1-3 for diagnostic purposes was not thresholded; all weights were used.

Permutation tests. To accomplish Step 5, we permuted the data 5,000 times, repeating the cross-validated LASSO-PCR analysis for each permuted dataset. The correlation between predicted and observed pain should be symmetrically distributed around zero if the procedure is unbiased, and this was tested and confirmed (FIG. 6D). In addition, the mean prediction error and predicted pain-observed pain correlation were far lower and higher, respectively, for the correct permutation (P<0.001 for both; FIG. 6D), demonstrating that the prediction results were far better than what would be expected by chance.

The following analyses examine several methodological aspects of the study, and demonstrate that a) head movement is not induced by thermal stimulation and does not drive pain-predictive results; and b) the time course of the signature response tracks pain experience more closely than the time course of noxious heat itself.

### Head Movement Analyses

In Study 1, to assess whether noxious thermal stimulation caused head movement, we quantified relationships between head movement and time within trial (anticipation, stimulation, and rating periods). We estimated head movement by taking the absolute successive differences between motion estimates from rigid-body image realignment during preprocessing. For each of the six directions of potential movement (lateral, anterior-posterior, and inferior-superior translation and roll, pitch, and yaw), movement was highest at the onset of the pain-predictive cue, but was still within standard tolerances even for the worst movement direction (<0.08 mm/0.06 degrees; FIG. 4A/B). Movement dropped within a few seconds to low levels, and stayed low throughout the stimulation epoch without responding to heat onset or offset. We also averaged head movement during the stimulation epoch as a function of stimulus temperature. Mixed-effects regression analyses revealed no significant relationships between temperature and head movement for any parameter (FIG. 4C/D). Effect sizes ranged from Z=0.17-0.92, all P>0.10. Similar results were obtained for other studies.

We also quantified the degree to which head movement and the inclusion of movement-related covariates impacted the sensitivity/specificity analyses. If pain is correlated with head movement, including head movement-related covariates should reduce performance in discriminating pain from other conditions. Conversely, if it is unrelated, controlling for head movement may increase discrimination accuracy by removing noise in the fMRI data. Across the six analyses of sensitivity/specificity reported for Study 1 (Pain vs. Low pain, Pain vs. Anticipation, and Pain vs. Pain Recall for each of pain/no-pain discrimination and forced-choice discrimination cases), effect sizes were moderately larger when controlling for head movement as described above (difference in dₐ=0.03-0.83, mean=0.49). Similar results were obtained for other studies.

### The Time Course of Signature Response

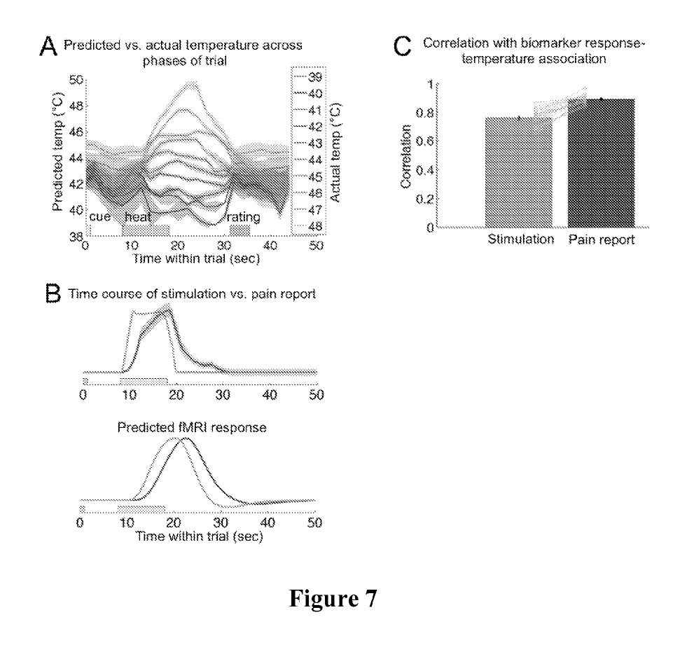

To examine the time course of the signature response during thermal stimulation and further assess the relationship with pain vs. heat sensation across time, we reconstructed signature response every 2 sec during the various phases of the stimulation trials: anticipation of pain, pain experience, pain judgment, and rest (FIG. 7). Signature response rose during the application of heat and monotonically tracked the actual temperatures, but did not respond to anticipatory cues or post-pain decision-making periods, demonstrating specificity to the time period when pain was experienced. In addition, stimulus delivery and subjective pain follow different time courses due to temporal summation (Koyama Y, et al. Pain 2004; 107:256-66; Apkarian A V, et al. J Neurophysiol 1999; 81:2956), permitting a test of which correlates more highly with signature response. We estimated the time course of subjective pain during heat epochs in a separate sample (N=12), and convolved that time course with the canonical SPM hemodynamic response function to obtain a prediction based on expected moment-by-moment pain experience (purple in FIG. S4B). We contrasted that with a model in which the time course of stimulation itself was convolved with the canonical SPM hemodynamic response function to obtain a prediction based on moment-by-moment heat intensity.

We estimated the slope of the relationship between signature activity and temperature at each time point for each participant. Correlation between the time course of signature temperature effects (slopes) and predicted fMRI responses were higher for the pain report predictor than the stimulation time course for every individual tested (r=0.89±0.007 vs. r=0.76±0.01, respectively; P<0.001; FIG. S4C). These results further suggest specificity to pain experience rather than general salience, somatic sensation, or decision processes.

### Experimental Design:

We delivered randomized sequences of thermal stimuli of varying intensities to participants' left forearms (‘trials’) during fMRI scanning with a 1.5 T General Electric scanner. Participants experienced 12 trials at each of four intensities calibrated for each individual: innocuous warmth (Level 1 on a 10-point visual analogue scale [VAS]; 41.0±1.9° C.) and three levels of painful heat (Levels 3, 5, and 7: 43.3±2.1° C., 45.4±1.71° C., and 47.1±0.98° C.). Each trial consisted of a warning cue and anticipation period (8 sec), stimulation (10 sec), and a pain recall/rating period (4 sec), with rest intervals pre- and post-recall.

### Deriving the Signature:

We used a machine-learning based regression technique, LASSO-PCR (least absolute shrinkage and selection operator-regularized principal components regression; Wager T D, et al. J Neurosci 2011; 31:439-52), to predict pain reports from fMRI activity. We selected relevant brain areas a priori using the Neurosynth meta-analytic database (Yarkoni T, et al. Nature Methods 2011) as explained in detail above, and averaged brain activity for each intensity level within each participant (Baliki M N, et al. J Neurophysiol 2009; 101:875-87; Lindquist M A, et al. NeuroImage 2009; 45:S187-S98; Wager T D, et al. Science 2004; 303:1162-7). We used the values within each 2×2×2 mm ‘voxel’ in the a priori map to predict continuous pain ratings, using leave-one-subject out cross-validation (see below). The result was a spatial pattern of regression weights across brain regions, which can be prospectively applied to fMRI activity maps from new individual participants. Application of the signature to an activity map (for example, a map obtained during thermal stimulation) yields a scalar response value, which constitutes the predicted pain for that condition. We used permutation tests to obtain unbiased estimates of accuracy, and bootstrap tests to determine which brain areas made reliable contributions to prediction. As described below, stimulation did not elicit head movement, and head movement estimates did not predict pain.

### Sensitivity and Specificity:

We assessed the signature's sensitivity and specificity to pain for two kinds of decisions. In ‘pain/no pain’ discrimination, the signature response values (i.e., the strength of expression of the signature pattern) for one condition are compared to a criterion threshold, with supra-threshold responses classified as painful. Receiver operating characteristic (ROC) plots trace the sensitivity/specificity tradeoff at different thresholds, and the threshold that minimizes overall decision errors is reported (Table 1). In forced-choice discrimination, two activation maps from the same individual are compared, and the image with the higher overall signature response (i.e., the stronger expression of the signature pattern) is classified as more painful. Forced-choice tests are particularly suitable for fMRI because they are ‘threshold-free’. Hence, they do not require people to use the pain reporting scale in the same way, and do not require the scale of fMRI activity to be the same across scanners. In this test, sensitivity, specificity, positive predictive value, and decision accuracy are equivalent.

### Results:

The neurologic signature included significant positive weights in regions including bilateral dpINS, S2, aIns, ventrolateral and medial thalamus (vlThal/mThal), hypothalamus, and dACC (q<0.05 false discovery rate [FDR]-corrected; FIG. 1A and Table 2), consistent with views of pain as a distributed process. In a leave-one-participant-out cross-validation test, the neurologic signature accurately predicted continuous pain ratings, with an average error of 0.96±0.33 (S.D.) units and a prediction-outcome correlation of r=0.74.

The signature response increased nonlinearly with stimulus intensity during thermal stimulation, but as expected, was uniformly low for anticipation and pain recall periods (FIG. 1B). To test discrimination of painful versus non-painful warmth, we compared painful conditions (>45° C., which activates specific nociceptors, and above the median pain report) vs. warm conditions (<45° C. and below median pain). Sensitivity and specificity in pain/no pain discrimination were 94% or greater for comparisons of pain versus non-painful warmth, pain versus anticipation, and pain versus pain recall (Table 1).

Forced-choice tests showed 100% sensitivity/specificity for all three comparisons (Table 1), indicating that signature response was always higher for painful stimulation than anticipation or recall within an individual. In addition, the signature discriminated relative differences in pain, with sensitivity/specificity≧93% when pain ratings differed by ≧2 units on the 9-point VAS scale. Thus, the neurologic signature was sensitive and specific to pain, with better performance in the forced-choice test.

TABLE 2
Peak coordinates from the machine learning analysis in Study 1.

| Name | x | y | z | mm³ | Z | Name | x | y | z | mm³ | Z |
|---|---|---|---|---|---|---|---|---|---|---|---|
| Thermal pain: Positive predictive weights | | | | | | Thermal pain; negative predictive weights | | | | | |
| Vermis (CBLM) | 2 | −53 | −20 | 486 | 3.35 | R ITC | 47 | −62 | −8 | 432 | −3.35 |
| R Ant/MidINS | 38 | 4 | 4 | 2241 | 3.35 | L Fusiform | −40 | −56 | −17 | 81 | −3.35 |
| | | | | | | gyrus | | | | | |
| L Superior | −40 | −11 | −8 | 162 | 3.35 | L Inferior | −40 | −80 | −11 | 378 | −3.35 |
| temporal gyrus | | | | | | Occipital | | | | | |
| | | | | | | gyrus | | | | | |
| R Calcarine gyrus | 8 | −89 | −5 | 189 | 3.35 | L Inferior | −34 | −65 | −8 | 162 | −3.35 |
| (BA17) | | | | | | Occipital | | | | | |
| | | | | | | gyrus | | | | | |
| R vlThal | 14 | −17 | 1 | 405 | 3.35 | L Inferior | −22 | −98 | −5 | 81 | −3.35 |
| | | | | | | Occipital | | | | | |
| | | | | | | gyrus (BA18) | | | | | |
| L midINS | −37 | 4 | 4 | 810 | 3.35 | vmPFC | 8 | 37 | 1 | 405 | −3.35 |
| Hypothal | 2 | −5 | 1 | 216 | 3.35 | L Middle | −55 | −41 | 4 | 567 | −3.35 |
| | | | | | | temporal | | | | | |
| | | | | | | gyrus | | | | | |
| L vlThal | −13 | −17 | 1 | 81 | 3.04 | L IFG | −52 | 25 | 4 | 162 | −3.35 |
| R frOP/temporal | 59 | 4 | 1 | 189 | 3.35 | R Inferior | 38 | −83 | 4 | 81 | −3.16 |
| pole | | | | | | Occipital | | | | | |
| | | | | | | gyrus | | | | | |
| L dpIns/SII | −40 | −20 | 13 | 270 | 3.35 | R Heschi's | 41 | −26 | 10 | 162 | −3.35 |
| | | | | | | Gyrus | | | | | |
| R dpINS | 41 | −17 | 13 | 324 | 3.35 | R Middle | 32 | −77 | 19 | 216 | −3.35 |
| | | | | | | Occipital | | | | | |
| | | | | | | Gyrus | | | | | |
| R SII | 59 | −17 | 15 | 162 | 3.04 | R Middle | 32 | −77 | 34 | 270 | −3.35 |
| | | | | | | Occipital | | | | | |
| | | | | | | Gyrus | | | | | |
| LTPJ (Superior | −64 | −32 | 22 | 216 | 3.35 | PCC/precuneus/ | −1 | −35 | 49 | 513 | −3.35 |
| temporal gyrus) | | | | | | paracentral | | | | | |
| | | | | | | lobule | | | | | |
| dACC | 2 | 13 | 31 | 1917 | 3.35 | R SPL | 23 | −62 | 55 | 297 | −3.35 |
| R Supramarginal | 53 | −32 | 31 | 108 | 3.35 | L SPL | −19 | −65 | 51 | 189 | −3.35 |
| gyrus | | | | | | | | | | | |
| R IPL | 59 | −35 | 37 | 152 | 3.16 | R Middle | 35 | −89 | 4 | 513 | −3.35 |
| | | | | | | Occipital | | | | | |
| | | | | | | Gyrus | | | | | |

The signature map was thresholded at q < 0.05 FDR for interpretation, based on a bootstrap test with 5000 bootstrap samples.
Peak coordinates for positive and negative weights are listed in the left and right columns, respectively.
Coordinates are reported in standard Montreal Neurologic Institute space.
ACC, anterior cingulate cortex;
CBLM: cerebellum;
IFG, inferior frontal gyrus;
INS, insula;
IPL, inferior parietal lobule;
ITC, inferior temporal cortex;
OCC, occipital; frOP, frontal operculum;
PCC, posterior cingulate cortex;
PHCMP, parahippocampal cortex;
PFC, prefrontal cortex;
SMA, supplementary motor cortex;
SPL, superior parietal lobule;
STS, superior temporal sulcus;
Thal, thalamus;
TPJ, temporal-parietal junction;
mvPFC, ventromedial prefrontal cortex.
Prefixes: a, anterior; d, dorsal; l, lateral; m, medial; r, rostral; s, superior; v, ventral.

### Example 3

This example illustrates Study 2, which demonstrates that the neurologic signature predicts pain at the level of an individual.

### Participants:

Study 2 included 33 healthy, right-handed participants (Mage=27.9±9.0 years, 22 females). The sample consisted of 39% Caucasian, 33% Asian, 12% Hispanic, and 15% African American participants.

### Materials and Procedures:

### Thermal Stimulation and Pain Ratings

Thermal stimulation was delivered to locations on the left volar forearm that alternated between runs. Each stimulus lasted 12.5 seconds, with 3-second ramp-up and 2-second ramp-down periods and 7.5 seconds at target temperature. Trials at six discrete temperatures were administered (level 1: 44.3° C., level 2: 45.3° C., level 3: 46.3° C., level 4: 47.3° C., level 5: 48.3° C., level 6: 49.3° C.). After each stimulus, participants rated explicitly whether it was painful or not. If they rated it as non-painful, they were then prompted to rate warmth intensity on a 100-point VAS anchored with “no sensation at all” and “very warm but not yet painful.” If they rated it as painful, they rated pain intensity on a 100-point VAS anchored with “no pain” and “worst imaginable pain.”

fMRI Task Design

FMRI images were acquired during 10 functional runs. Runs 1, 2, 4, 8 and 9 were “standard” runs, during which were delivered a total of 11 stimulations from each of levels 1-5, for a total of 55 stimuli. Transitional frequencies were counterbalanced so that each temperature was preceded twice by each of the five temperatures and each run started with a different temperature. Different presentation orders were generated for each participant. On Runs 5-6 temperatures were increased one degree, with 4 stimuli at each of levels 2-6. During two additional runs (not analyzed here), participants were instructed on the use of mental imagery to modify pain.

Each trial consisted of a stimulus (12.5 sec), a 4.5-8.5 sec delay, a 4 sec painful/non-painful decision period (participants pressed the left or right button on the side of an MR-compatible trackball), a 7-sec continuous warmth or pain rating period (VAS ratings were made using the trackball and confirmed with a button-press), and 23-27 sec of rest. During both rest and stimulation, participants fixated on a cross presented on-screen.

fMRI Acquisition and Analysis

Imaging Acquisition.

Whole-brain fMRI data were acquired on a 3 T Philips Achieva TX scanner at the PICS Center. Structural images were acquired using high-resolution T1 spoiled gradient recall images (SPGR) for anatomical localization and warping to a standard space. Functional EPI images were acquired with TR=2000 ms, TE=20 ms, field of view=224 mm, 64×64 matrix, 3×3×3 mm voxels, 42 interleaved slices, parallel imaging, SENSE factor 1.5. Runs lasted between 6:22 and 6:58 (191 or 209 TRs). Stimulus presentation and data acquisition were controlled using E-Prime.

Preprocessing and Analysis.

Image preprocessing and analysis were performed as described under General fMRI Processing above. First-level GLM analyses for each participant included regressors for stimulation periods for each of the 6 levels and the 11-sec rating periods, linear drift across time within each run, and indicator vectors for outliers and head movement as described above. The signature pattern from Study 1 was used to estimate the signature response for each participant in each condition, and these values were used in binary classification analyses.

To assess classification performance for painful vs. non-painful trials, we averaged signature responses for non-painful and painful trials, and subjected these average responses to sensitivity/specificity analyses. Because this study was collected on a different scanner with a higher field strength, signature responses were on a different scale and a different classification threshold was determined for pain/no-pain classification. Forced-choice analyses are threshold-free and do not require this adjustment.

Regression Models.

In a second model, we included separate regressors for each individual trial, and applied the signature pattern from Study 1 to estimate the signature response for each individual trial. We used these values in mixed effects regression models predicting pain and temperature. Both warmth ratings and pain ratings were very sensitive to temperature increases: Pain ratings increased 20.8±12.9 (SD) units/° C., and warmth ratings increased 17.7±12.7 units/° C.

In the regression analyses, we tested models in which we assessed performance in predicting pain controlling for temperature. To completely control for temperature, we included covariates that controlled for all possible pairwise differences between temperatures (level 6 vs. 5, 5 vs. 4, 4 vs. 3, 3 vs. 2, and 2 vs. 1), thus controlling for temperature estimated in a nonparametric fashion, without assuming linearity. This analysis removed much of the variation in pain report (as most of the variance was caused by temperature), but served as a test of whether signature responses predicted pain even when completely accounting for the effects of heat itself.

### Experimental Design:

We delivered randomized sequences of thermal stimuli of varying intensities to participants' left forearms (trials′) during fMRI scanning with a 1.5 T General Electric scanner. Participants experienced 75 total trials across six temperatures (44.3-49.3° C. in 1-° C. increments on the left forearm). After each trial, participants judged whether the stimulus was painful, and then judged warmth or pain intensity on a 100-point VAS. Ratings were coded as 0-99 for non-painful and 100-200 for painful events.

### Predicting Pain in an Independent Sample:

We tested the neurologic signature identified in Study 1, with no further model fitting, for prediction of pain in individual subjects using data from a different scanner. We also estimated activity maps and signature responses for individual trials, allowing us to use mixed-effects regression models to test the relationship between neurologic signature responses and intensity judgments during trials involving painful and non-painful stimuli.

### Results:

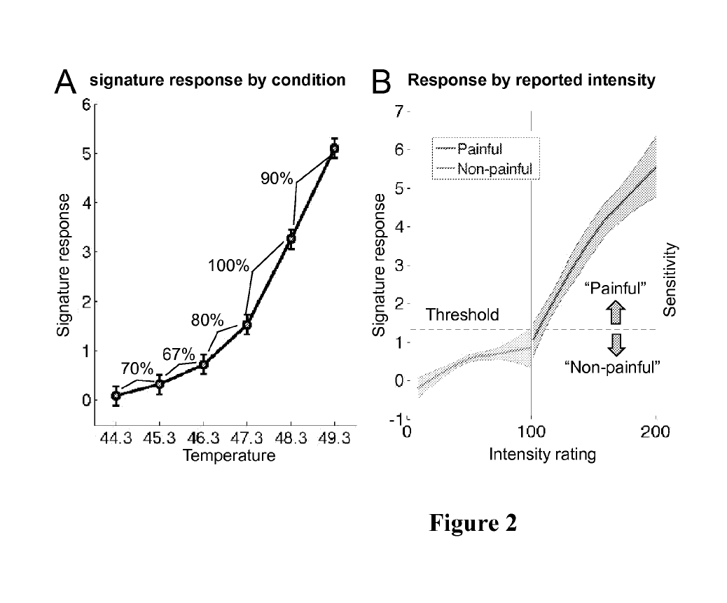

Signature response increased monotonically across the six temperatures (Model 1; FIG. 2A), with an expected nonlinear increase with temperature, and correlated with both pain reports (r=0.73) and stimulus temperature (r=0.65). Signature responses increased with subjective intensity on a continuum across painful and non-painful events (FIG. 2B), consistent with contributions by co-localized wide dynamic range and nociceptive-specific neurons (Craig A D, et al. J Neurophysiol 2001; 86:1459-80; Dong W K, et al. 1989; 484:314-24; Kenshalo D R, et al. J Neurophysiol 2000; 84:719-29). However, mixed-effects regression analyses showed that signature response increased more strongly with pain intensity than warmth intensity ratings (\hat{β}=0.66, t=2.58, P=0.02; FIG. 2B). On painful trials, the neurologic signature strongly predicted pain intensity (\hat{β}=0.20, t=6.84, P<0.001), even when controlling for linear and nonlinear effects of temperature (\hat{β}=0.13, t=4.51, P<0.001). On non-painful trials, the neurologic signature weakly predicted warmth intensity (\hat{β}=0.06, t=2.04, P=0.08) and did not predict warmth intensity after adjusting for temperature (\hat{β}=0.05, t=1.30, P=0.22). These results suggest that the signature is related principally to the subjective sensation of pain, but also reflects the overall intensity of somatic stimulation to some degree.

To assess discrimination performance, we averaged the neurologic signature response for painful (rating≧100, average 138) and non-painful (rating<100, average 60) conditions for each individual. Because the scanner field strengths differed for Studies 1 and 2 (1.5 T vs. 3.0 T), we estimated a new criterion threshold of 1.32 for painful vs. non-painful events (cf. 1.40 in Study 1). Average signature response accurately discriminated painful from non-painful conditions with 93% sensitivity and specificity in the pain/no-pain test (95% confidence interval [CI], 84-100% for both), and 100% sensitivity/specificity (CI: 100-100%) in the forced-choice test (Table 1, supra). Signature response also discriminated clearly painful conditions from those near the pain threshold (mean rating=150 vs. 98) with 88% sensitivity (CI: 77-97%) and 85% specificity (CI: 72-95%) in the pain/no-pain test and 100% sensitivity/specificity in the forced-choice test. However, signature response also discriminated intense versus mild non-painful warmth (see Table 1, supra). Thus, demonstrating hyperalgesia or allodynia should require positive results in both the pain/no-pain and forced-choice tests.

Finally, tests of forced-choice discrimination across painful temperatures showed good performance; tests across non-painful temperatures showed poor performance, supporting the use of the signature to assess nociceptive responses. Sensitivity/specificity was 90% (CI: 81%-97%) for 49.3° C. vs. 48.3° C., with only 4 trials delivered at 49.3° C., and 100% for 48.3 vs. 47.3° C., with 15 trials in each condition. However, performance dropped to near-chance levels at low temperatures (FIG. 2A).

### Example 4

This example illustrates Study 3, which demonstrates that the neurologic signature is specific and is able to discriminate between physical pain and social pain.

### Participants:

Study 3 included 40 participants (aged 20.8±2.6 years, 21 females). Forty right handed, native English speakers (21 females, M_{age} 20.78, SD=2.59) gave informed consent. All participants experienced an unwanted romantic relationship break-up within the past six months (M=2.74 months; SD=1.70 months), and indicated that thinking about their break-up experience led them to feel rejected. All participants scored above the midpoint on a 1 (not at all rejected) to 7 (very rejected) scale that asked them to rate how rejected they feel when they think about their rejection experience (M=5.60, SD=1.06). The sample consisted of 60% Caucasian, 20% Asians, 10% African Americans, and 10% other ethnicities. Data were collected between 2007-2008. Data on the basic group activation maps for physical and social pain contrasts were published previously (Kross E, et al. PNAS 2011; 108:6270-5), but the analyses and substantive conclusions were different from and complementary to those reported here.

### Materials and Procedures:

### Social Pain Stimuli

The social rejection task was modeled after (a) fMRI research that used photographs provided by participants to elicit powerful emotions, including maternal love, romantic love, and rejection and (b) behavioral research indicating that cueing people to recall autobiographical rejection experiences is an effective way of reactivating social rejection related distress. The stimuli for this task consisted of: (a) a headshot photograph of each participant's ex-partner and a same gendered friend with whom they shared a positive experience around the time of their break-up (M=2.46 months; SD=1.70 months), and (b) cue phrases appearing beneath each photograph which directed participants to focus on a specific experience they shared with each person.

All photographs were cropped so that the total area of the photograph taken up by the face was constant across ex-partner and friend images (t=1.42, P=0.16). To be sure that the photographs participants provided were matched in terms of picture quality, we had a group of ten individuals who were blind to the study goals and hypotheses rate the picture quality of each photograph. Ex-partner and friend photographs did not differ significantly on this dimension (t=1.32, P=0.20). Judges also rated the attractiveness level of the individuals depicted in ex-partner and friend photos, which also did not differ significantly (t=0.89, P=0.38).

When participants viewed the photograph of their ex-partner during the social rejection task they were instructed to think about how they felt during their specific break-up experience; when they viewed the photograph of their friend they were instructed to think about how they felt during their recent positive experience with that person. To help participants focus on these specific experiences during the task we included a short cue phrase beneath each photograph (e.g., “rejected by Marc”; “party with Ted”). Participants generated these cue phrases on their own, prior to the day of scanning using a procedure developed in prior research (Kross E, et al. Biol Psychiatry 2009; 65:361-6). Specifically, they first wrote about their specific break-up experience with their ex-partner and their specific positive experience with their friend. Subsequently, they were asked to create a cue phrase that captured the gist of their experience. They were reminded of the cues they generated and their break-up experiences on the day of scanning following established procedures (Maihofner C, et al. Neurology 2006; 66:711-7).

### Physical Pain Stimuli

As in Study 1 and prior research (Rish I, et al. Brain Informatics 2010; Wager T D, et al. Science 2004; 303:1162-7; Wager T D, et al. Science 2004; 303:1162-7; Wager T D, et al. PNAS 2007; 104:11056-61), a calibration procedure was used to select heat intensities that participants judged to be non-painful (“warm,” Level 2 on a 10-point scale) vs. near the limit of pain tolerance (“hot,” as close as possible to Level 8 on a 10-point scale, though intensity was capped at 48° C.). The mean low temperature for the sample was 39.9° C. (SD=2.76° C.); the mean high temperature was 46.6° C. (SD=1.72° C.). In the scanner, participants rated both physical and social pain on a 5-point scale using a five-button unit under their right hand, with lower numbers reflecting more distress.

### Task Training

Prior to scanning, the experimenter walked participants through each step of the social rejection task (referred to as the “photograph” task to participants) and the physical pain task (referred to as the “heat” task to participants). They were told that that during the “photograph” task they would see the photographs of their ex-partner and friend. The experimenter explained that beneath each photograph the cue-phrases they generated earlier would appear. When they saw each photograph they were asked to look directly at it and think about how they felt during the specific experience associated with the cue-phrase. Thus, when participants viewed the photograph of their ex-partner they were directed to think about how they felt during their break-up experience with that person; when they viewed the photograph of their friend they were directed to think about how they felt during their positive experience with that person. During the physical pain task, participants were instructed to focus on the fixation cross that appeared on the screen during the trials, and think about the sensations they experienced as the thermode on their arm heated up. They were then instructed how to rate their affect after each type of trial, and how to perform the visuospatial control task.

fMRI Acquisition and Analysis

Acquisition

Whole-brain functional data were acquired on a GE 1.5 T scanner at the PICS Center (the same scanner used in Study 1) in 24 contiguous axial slices (4.5 mm thick, 3.5×3.5 mm in-plane resolution) parallel to the anterior commissure-posterior commissure (AC-PC) line with a T2*-weighted spiral in out sequence (repetition time [TR]=2000, echo time [TE]=40, flip angle=84, field of view [FOV]=22.4) in 4 runs of 184 volumes each (368 sec each). Structural data were acquired with a T1-weighted spoiled gradient recalled echo scan (180 slices, 1 mm thick, in-plane resolution 1×1 mm; TR=19, TE=5, flip angle=20, FOV=25.6).

Analysis: Image preprocessing and analysis were performed as described under General fMRI Processing above, except that functional data were smoothed with a 6 mm FWHM Gaussian kernel after spatial warping and prior to analysis (as done in a prior publication on these data; Meier M L, et al. Journal of clinical periodontology 2012). First-level GLM analyses for each participant included regressors for Rejector photos, Friend photos, Hot (painful) stimulation, and Warm (peri-pain threshold) stimulation periods, as well as covariates for the 5 sec affect rating periods for each condition and movement and outlier covariates for each run. The signature pattern from Study 1 was used to estimate the signature response for each participant in each condition, and these values were used in binary classification analyses.

### Experimental Design:

We delivered randomized sequences of thermal stimuli of varying intensities to participants' left forearms (‘trials’) during fMRI scanning with a 3 T Phillips scanner. Participants experienced 32 trials, consisting of eight trials with each of four stimulus types. We delivered noxious heat (‘Painful’, 46.6±1.7° C.) and near pain-threshold warmth (‘Warm’, 39.9±2.8° C.) at individually calibrated temperatures. Each participant had recently experienced a romantic breakup and continued to feel intensely rejected. Participants viewed an image of their ex-partner (‘Rejector’ trials, which elicit social pain (MacDonald G, Leary M R. Psychol Bull 2005; 131:202-23)) and an image of a close friend (‘Friend’ trials) during scanning.

### Testing for Specificity:

We applied the signature to activation maps resulting from physical sensation (Painful and Warm conditions) and from viewing images related to ‘social pain’ (Rejector and Friend conditions).

### Results:

[Rejector—Friend] and [Pain—Warm] comparisons yielded comparable levels of self-reported negative affect and activated overlapping portions of many pain intensity-related regions, including bilateral aIns, mThal, SII and dpINS, providing a good substrate for a test of specificity.

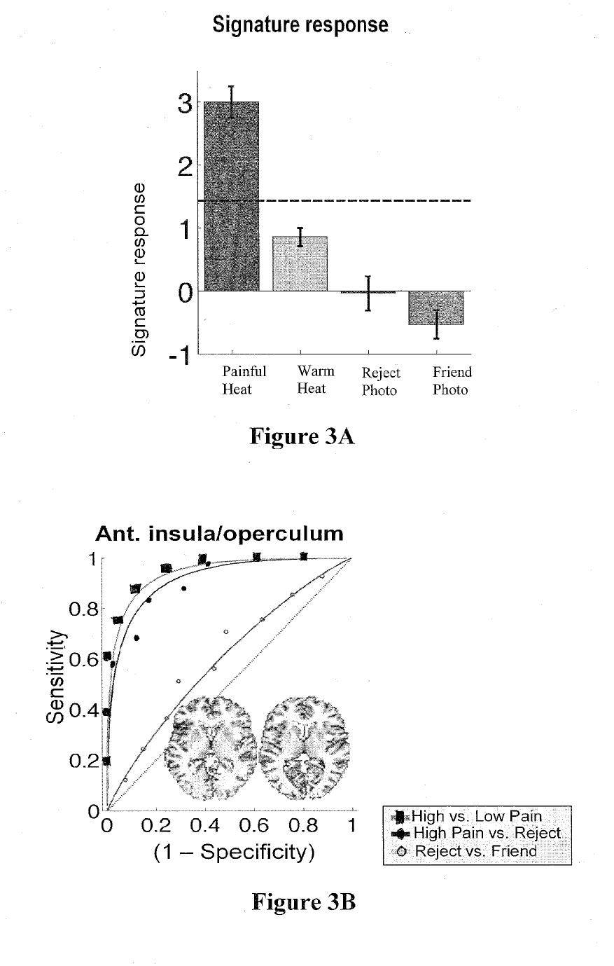

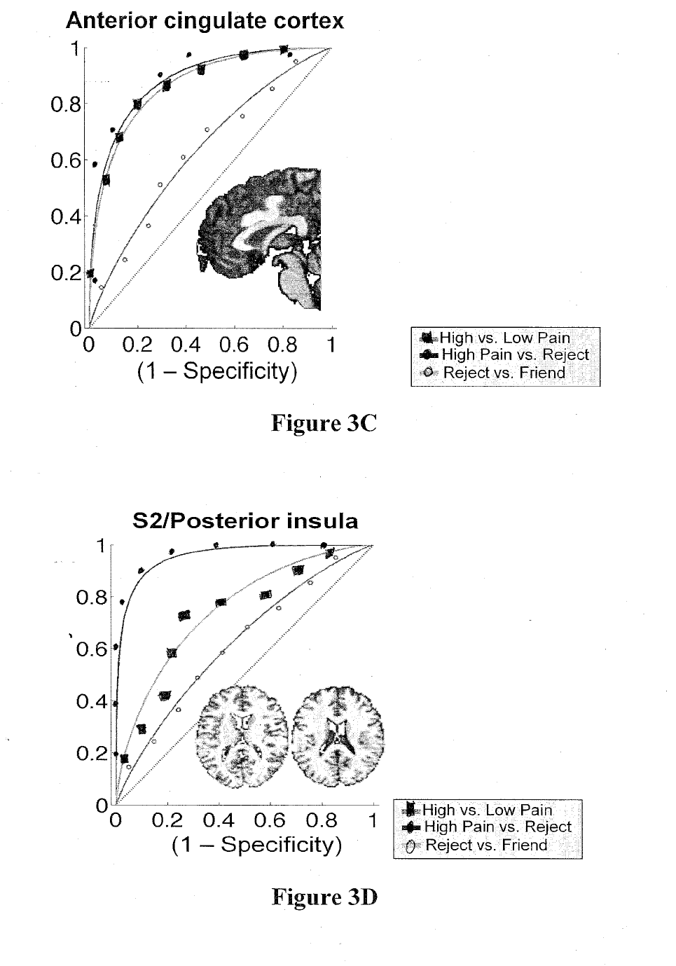

The neurologic signature response was substantially stronger for physical pain than for any of the other conditions (Warm, Rejector, and Friend; FIG. 3A) and predicted pain ratings (r=0.68, P<0.001, with an average prediction error of 0.84 units). As in Study 1, the signature response predicted intensity ratings for noxious (r=0.44, P<0.01), but not innocuous (r=0.02, P>0.90), stimuli. Using the threshold derived from Study 1, pain/no-pain discrimination had 85% (CI: 76-94%) sensitivity and 78% (CI: 67-89%) specificity for Pain versus Warm and 93% (CI: 86-98%) sensitivity/specificity in forced-choice discrimination, with comparable performance for Pain versus Rejection (Table 1, P<0.001 for all). Discrimination of Rejector versus Friend conditions was no better than would be expected by chance (Table 1, supra).

This observed specificity may be driven by a) fine-grained differences in activity patterns in regions activated by both physical and social pain, consistent with the notion that different neural populations code for different affective events, or b) differential activation of modality-specific regions (e.g., S2 for heat versus occipital cortex for pictures). If (a) holds, the pattern of activation rather than the overall level of activation of a region is the critical agent of discrimination. To test these alternatives, we assessed the neurologic signature response in the dACC, aIns, and dpINS patterns individually (FIG. 3B-D). Each region was activated by social pain ([Rejector versus Friend]) overall. However, in each region, the signature response reliably discriminated Pain from Warm and Rejector conditions (average forced-choice sensitivity/specificity=78%; Table 3) and was at chance for Rejector versus Friend (average sensitivity/specificity=58%), suggesting that the pattern within these regions is critical for predicting pain.

TABLE 3
Forced-choice classification performance across studies.

| | Discrimination | Effect size | | Binomial test |
|---|---|---|---|---|
| Forced-choice discrimination test | Sens./Spec./PPV^{h} | AUC | dₐ | P-value |
| Study 1 | | | | |
| Painful vs. Warm^{a} | 100% (100-100%) | 1.00 | 4.88 | P < 0.001 |
| Pain vs. Anticipation | 100% (100-100%) | 1.00 | 3.92 | P < 0.001 |
| Pain vs. Pain Recall | 100% (100-100%) | 1.00 | 2.29 | P < 0.001 |
| Conditions different by 3+ VAS units^{f} | 100% (100-100%) | 1.00 | 3.91 | P < 0.001 |
| Conditions different by 2-3 VAS units | 93% (84-100%) | 0.97 | 2.17 | P < 0.001 |
| Conditions different by 1-2 VAS units | 86% (76-95%) | 0.86 | 1.15 | P < 0.001 |
| Conditions different by 0.5-1 VAS unit | 69% (50-90%) | 0.80 | 0.99 | P = 0.26 |
| Study 2 | | | | |
| Painful vs. Warm^{c} | 100% (100-100%) | 1.00 | 3.12 | P < 0.001 |
| Painful (>125) vs. near-threshold (75-125)^{e} | 100% (100-100%) | 1.00 | 2.77 | P < 0.001 |
| High (50-100) vs. low (0-50) warmth | 100% (100-100%) | 1.00 | 2.18 | P < 0.001 |
| 49.3^{g} vs. 48.3° C. | 90% (81%-97%) | 0.93 | 1.71 | P < 0.001 |
| 48.3 vs. 47.3° C. | 100% (100-100%) | 1.00 | 2.00 | P < 0.001 |
| 47.3 vs. 46.3° C. | 80% (67%-91%) | 0.82 | 0.96 | P = 0.001 |
| 46.3 vs. 45.3° C. | 67% (53%-81%) | 0.77 | 0.77 | P = 0.10 |
| 45.3 vs. 44.3° C. | 70% (56%-83%) | 0.66 | 0.43 | P = 0.04 |
| Study 3 | | | | |
| Painful vs. Warm | 93% (86-98%) | 0.97 | 2.08 | P < 0.001 |
| Painful vs. Rejector Photo | 95% (89-100%) | 0.98 | 2.09 | P < 0.001 |
| Rejector Photo vs. Friend Photo | 56% (43-69%) | 0.66 | 0.49 | P = 0.53 |
| Study 4 | | | | |
| Hot vs. Warm, pre-drug | 90% (79-100%) | 0.97 | 1.76 | P < 0.001 |
| Hot vs. Warm, on-drug | 76% (61-90%) | 0.84 | 1.08 | P < 0.05 |
| Hot pre-drug vs. on-drug | 76% (60-92%) | 0.84 | 1.08 | P < 0.05 |

^{a}Painful conditions were defined as those >44.5° C. and >5.80 average VAS units, and Warm as <44.5° C. and <3.34 VAS units.
b: Study 2 was conducted on a scanner with a different field strength (3T), so a new threshold was estimated.
^{c}Participants made painful vs. non-painful judgments on each trial.
d: The threshold derived from Study 1 was applied.
^{e}Continuous, 100-point VAS ratings for pain or warmth intensity (0-99 for warmth, 100-200 for pain).
^{f}Visual analogue scale (VAS) ratings on a continuous, 9-point scale.
^{g}Only 4 trials were included at 49.3° (cf. 11 trials for 44.3° and 15 trials for other conditions.)
^{h}For two-choice (forced-choice) discrimination, the decision threshold (for the difference between pairs) is 0, and the sensitivity, specificity, and positive predictive value (PPV) are the same, and are equal to the decision accuracy.
AUC: Area under the Receiver Operating Characteristic curve, a threshold-independent measure of performance; chance is 0.5.
PPV: Positive predictive value.
da: Discriminability, a measure of effect size under a Gaussian model.
Performance varies to some degree based on the number of trials per subject averaged to form condition maps in each study.

### Example 5

This example illustrates Study 4, which shows that the neurologic signature responds to treatment with a known analgesic, remifentamil.

### Participants:

Study 4 included 21 participants (aged 24.7±4.2 years, 11 females). Twenty-one healthy, right-handed participants completed the study (M_{age}=24.7±4.18 years, 11 females). The sample consisted of 40% Caucasian, 15% Asian, 30% Hispanic, and 15% African American participants. Data on dissociable drug effects and expectancy effects were published previously (Atlas L Y, et al. J Neurosci 2012; 32:8053-64), but the analyses and substantive conclusions were different from and complementary to those reported here.

### Materials and Procedures:

### Thermal Stimulation and Pain Ratings

FMRI images were acquired during 2 functional runs of 6 blocks each (6 trials/block, 64 trials), with 30-second breaks between blocks, during which an experimenter rotated the thermode location. The thermode was placed on a different skin site for each block, and skin sites were stimulated in the same order on each run. Temperatures were selected for each individual based on a thermal pain calibration procedure (see above, “Thermal stimulation and pain ratings”), and thermal stimulation alternated between stimuli calibrated to elicit low pain (Level 2; M=41.16° C., SD=2.64) and high pain (level 8; M=47.05° C., SD=1.69).

### Remifentanil Administration and Experimental Design

During fMRI scanning, participants received remifentanil hydrochloride (Ultiva; Mylan Institutional) intraveneously under two conditions (‘runs’): Open administration, in which participants were fully informed about the drug infusion, and Hidden administration, during which participants were told they would receive no drug. Remifentanil administration proceeded identically in both runs. Run order was counterbalanced, such that half the participants received the Open run first, and half the Hidden run first, in a crossover design. Participants received remifentanil at doses individually selected to elicit pain relief without sedation, based on a pre-experiment dosing procedure. The average dose administered was 0.043 μg/kg/min (SD=0.01). Remifentanil infusion began after the first block (before trial 7), and infusion proceeded steadily throughout blocks 2-4, for the next 18 trials. Infusion was stopped and a washout period began following the fourth block, and anatomical images were acquired between runs to allow additional time, so that the brain concentrations of remifentanil were negligible at the start of the next run.

Thirty-six trials were administered in each run, 18 with painful heat and 18 with non-painful warmth. Pain and warm trials alternated, with order (pain first or warm first) counterbalanced across participants in a crossover design. At the start of each trial, participants heard an auditory tone (an orienting cue) and saw the words “warm” or “hot” on the screen for 3 s. Following a 7-13 s jittered anticipation interval (M=10.16 s, SD=2.64), participants felt heat from the thermode at temperatures calibrated to elicit either low or high pain (1.5 s ramp-up, 7 s at peak, 1.5 s ramp-down). This was followed by a 9-15 s rest interval (M=11.67 s, SD=2.50), during which participants fixated on a cross. The words “How painful?” then appeared on the screen for 4-6 seconds above a 9-point visual analogue scale (VAS), accompanied by an orienting tone. As in Study 1, participants rated the intensity of the stimulus using an fMRI-compatible track-ball (Resonance Technologies, Inc.). The next trial began after 9-15 s (M=11.46 s, SD=2.57).

fMRI Acquisition and Analysis

Image Acquisition.

Whole-brain structural (T1-weighted SPGR) and EPI fMRI data were acquired on a 1.5 T GE Signa Twin Speed Excite HD scanner (GE Medical Systems) at Columbia University's Program for Imaging in Cognitive Science (PICS), as in Studies 1 and 3. (EPI; TR=2000 ms, TE=34 ms, field of view=224 mm, 64×64 matrix, 3.5×3.5×4.0 mm voxels, 28 slices). Each run lasted 33 minutes and 20 seconds (1000 TRs), divided into six blocks, with a brief pause between blocks 4 and 5 to prevent scanner overheating. Stimulus presentation and behavioral data acquisition were controlled using E-Prime software (PST Inc.).

Preprocessing.

Preprocessing was identical to that described in the General Methods, except that FSL software was used for realignment.

Analysis.

We used first-level (single-subject) GLM regression parameter estimates from our previously published study (Atlas L Y, et al. J Neurosci 2012; 32:8053-64) (but adjusted to 3×3×3 mm voxels), which maintained consistency in modeling of the events and drug effects across the previous report and this one. Full details of the model are provided in the previous publication, but in brief, we modeled effects of painful (Hot) and non-painful (Warm) stimulation in each of Open and Hidden runs with separate regressors. model drug effects across time, we used a pharmacokinetic model and parameter estimates based on age, weight, and sex (Minto C F, et al. Anesthesiology 1997; 86:10-23) and Minto C F, et al. Anesthesiology 1997; 86:24-33) to estimate the drug effect site concentration second-by-second during drug infusion. Those values were normalized to a peak amplitude of 1 and used to create a “parametric modulator” regressor for each condition, which is orthogonal to the average regressor across trials and estimates changes in heat-evoked responses across time that are linearly related to drug effect site concentration. To capture additional effects of expectations and other time-varying effects that do not follow the time-course of drug effects, we included an additional parametric modulator, which modeled the period of infusion vs. pre- and post-infusion baseline, orthogonalized to the drug effect site concentration regressor. Together, the regressors capture a range of modulatory effects across time, including drug effects based on the pharmacokinetic model.

To test Hot vs. Warm and drug effects on the signature response, we applied the signature pattern from Study 1 to each regression parameter estimate (\vec{β}ₘₐₚ) map to yield a single amplitude value (BR) for each regressor within each participant. The significance of the drug modulation effect on signature response was tested by conducting a t-test on the BR values for the drug effect site concentration regressor. To visualize the responses (FIG. 4), we reconstructed the fitted responses for Hot and Warm trials in each of Open and Hidden administration by multiplying the appropriate regressors in the design matrix X by BR for each participant. This yielded an overall fitted time course for each condition within each subject. To conduct analyses on pre-drug infusion and peak drug infusion trials, we constructed a GLM design matrix with regressors for each trial, and used it to estimate the amplitude of the fitted response on each trial. Estimates for pre-drug infusion trials were obtained by averaging across amplitudes for Trials 1-3 for each participant, and estimates for peak drug infusion trials were obtained by averaging amplitudes for Trials 10-12.

### Experimental Design:

We delivered randomized sequences of thermal stimuli of varying intensities to participants' left forearms (‘trials’) during fMRI scanning with a 1.5 T General Electric scanner. Participants received two intravenous infusions (‘runs’) of remifentanil, a potent μ-opioid agonist, during fMRI scanning. In an Open infusion run, participants knew they received remifentanil, and in a Hidden run, they were told that no drug was delivered. Remifentanil doses (0.043±0.01 μg/kg/min) were individually titrated to elicit analgesia without sedation, and we estimated the brain concentration of the drug across time using a pharmacokinetic model. Thirty-six trials—18 painful (47.1±1.7° C.) and 18 warm (41.2±2.6° C.)—were delivered during each of the two runs. Drug infusion began part-way through each run, after six trials, and ended after 24 trials. This design produced a continuously varying level of drug concentration across time within each run.

### Response to Analgesic Treatment:

We tested the effects of stimulus intensity (painful vs. warm), drug (remifentanil concentration), and psychological context (Open vs. Hidden) on the biomarker response. For each of the Open and Hidden runs, we estimated activation maps for painful stimulation, warm stimulation, and the magnitude of changes in each that followed the a priori time course of drug concentration from the pharmacokinetic model. Because drug concentration was continuous over time, binary classification of painful vs. warm conditions was performed on averages of three pre-drug trials vs. three trials at peak drug concentration.

### Results:

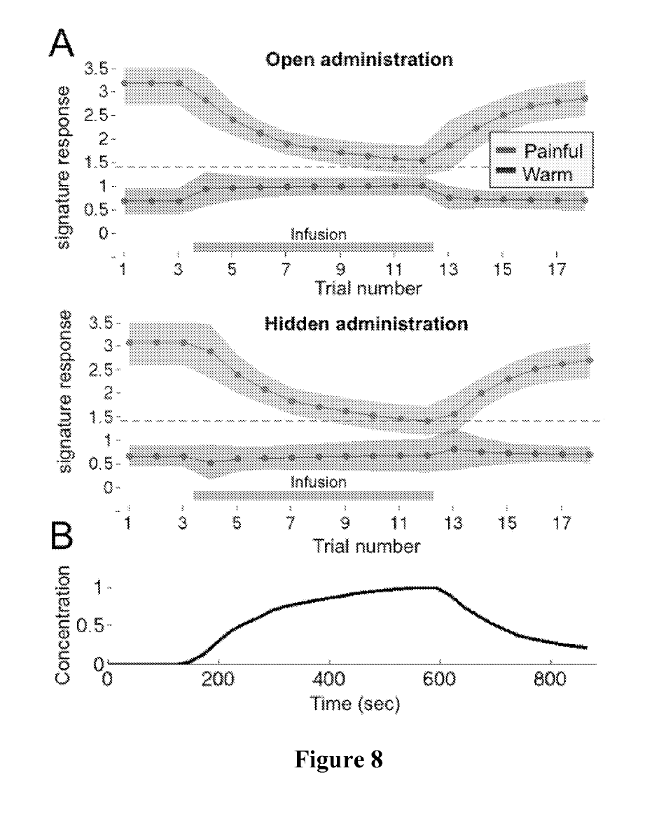

Before drug infusion, the signature response was greater for Painful versus Warm stimuli in both the Open and Hidden runs (t(20)=5.21 and 4.84, both P<0.001; FIG. 8). During infusion, the signature response was reduced in parallel with increases in the drug effect-site concentration (t(20)=−2.78 and −2.77 for open and hidden, both P=0.01). Remifentanil reduced the signature response by 53% at maximum drug concentration, with no differences across Open and Hidden runs (P=0.94). Painful vs. warm discrimination sensitivity/specificity was 90% (CI: 79-100%) in the forced-choice test, with 95% (CI: 86-100%) sensitivity and 62% (CI: 43-79%) specificity in the pain/no-pain test (P<0.001; see Table 1, supra). Lower accuracy was expected because pre-infusion signature responses in each condition were estimated from only 3 trials.

### Example 6

This example demonstrates that the Neurological Pain Signature (NPS) described in this disclosure is sensitive to changes in the intensity of a painful stimulus, but cannot be altered (increased or decreased) by training participants to imagine and think about pain differently.

In this study, we enrolled 30 human participants and 1) manipulated nociceptive input and 2) trained the participants in a cognitive regulation strategy in which they were taught to increase and decrease pain (in separate test blocks). The results demonstrate that cognitive regulation effects on pain were independent of the NPS response, providing crucial validation that the NPS is insensitive to some forms of cognitive intervention. While the cognitive regulation effects strongly influenced the participant's pain reports, they had no effects on the NPS. Cognitive regulation effects were mediated through a pathway connecting the nucleus accumbens (NAc) and ventromedial prefrontal cortex (vmPFC), establishing the existence of a second pathway that mediates cognitive effects on pain. This pathway was unresponsive to noxious input, but has been implicated in long-term pain and reward-related decision-making.

### Example 7

This example demonstrates that the Neurological Pain Signature (NPS) is sensitive to changes in the intensity of a painful stimulus in a new study conducted on a different scanner (a 3.0 T Siemens Tim Trio in Boulder, Colo.), but is not sensitive to the intensity of vicarious pain, the observation of pictures of others in pain. Thus, these data show the specificity of the NPS to physical pain. It also shows that the NPS tracks pain intensity across upper limb (arm) and lower limb (foot) body sites. Additionally, it shows that we can distinguish body-part specific brain activity patterns that can discriminate upper vs. lower limb pain with >90% accuracy in individual persons. We identified and tested multi-voxel fMRI activity patterns that track experienced and vicarious (observed) pain in specific body regions. The response in the original NPS signature was sensitive to pain on both hand and foot sites (hand: t(27)=9.08, p<0.0001, foot: t(27)=8.88, p<0.0001), demonstrating generalizabilty. It showed no response to vicarious pain. We also developed a vicarious pain signature (VPS) with cross-validated, multivariate pattern analyses that tracked the intensity of vicarious pain for both hand and foot sites (hand: t(27)=7.42, p<0.0001, foot: t(27)=10.44, p<0.0001). The VPS did not respond to somatic pain. Thus, the two types of pain engage fundamentally different circuits. Finally, support vector machine (SVM) classifiers could differentiate between pain on hand vs. foot with 93% accuracy on an individual-person basis.

### Example 8

This example demonstrates distinctiveness between biomarkers for pain versus those for aversive taste. Because pain and taste are both primary reinforcers represented in the insula, we hypothesized that they are confusable at the neural level. We trained separate classifiers to a) detect the intensity of aversiveness across pain (heat) and taste (quinine) modalities, and b) differentiate between pain and bitter taste stimulations. Preliminary results show distinct representations for thermal pain vs. aversive taste; classification was >90% accuracy on a per-individual basis.

### Example 9

This example demonstrates the use of supervised machine learning techniques to identify two distinct fMRI-based brain markers that were sensitive and specific to social pain (viewing ex-partners' photos) and somatic pain (painful thermal stimulations). In a study based on 60 human participants, two fMRI pattern-based markers were shown to be separately modifiable by social pain and somatic pain and uncorrelated with each other (r=−0.04 across classifier weights) even though there was substantial overlap in fMRI activity between two modalities of pain. The fMRI-based markers for social and somatic were accurate at the individual-person level (88% and 100%, respectively) and specific to each type of pain. These data show that it is possible to find brain activity patterns that track the intensity of negative emotional experiences, and that the NPS provided in this disclosure is specific to physical pain and does not respond to negative emotional experiences.

1. A method of detecting pain in a subject comprising:
a. applying a stimulus to the subject;
b. measuring brain activity of the subject in response to the stimulus using functional Magnetic Resonance Imaging (fMRI) and generating a brain map of the subject representing the brain activity of the subject; and
c. comparing the brain map of the subject to a neurologic signature map, wherein the neurologic signature map represents brain activity indicative of pain.

2. The method of claim 1, wherein the signature map comprises a fMRI pattern that is at least 70% identical to the fMRI patterns shown in FIG. 1A.

3. The method of claim 1, wherein the method comprises applying the signature map to the brain map of the subject to provide a response value.

4. The method of claim 1, wherein the method comprises analyzing similarities and dissimilarities between portions of the brain map of the subject and the corresponding portions of the signature map.

5. The method of claim 3, further comprising quantifying the pain in the subject based on the response value.

6. The method of claim 1, further comprising diagnosing a pain-related condition in the subject, wherein the condition is selected from the group consisting of hyperalgesia, allodynia, pain catastrophizing, fear of pain, chronic neuropathic pain, complex regional pain syndrome, reflex sympathetic dystrophy, post-stroke pain, fibromyalgia, inflammatory pain, and nociceptive pain.

7. The method of claim 1, further comprising administering an analgesic to the subject.

8. The method of claim 7, wherein the analgesic is selected based on the comparison between the brain map of the subject and the signature map.

9. The method of claim 7, wherein the dosage of the analgesic is selected based on the comparison between the brain map of the subject and the signature map.

10. The method of claim 1, wherein the comparing is done by computer.

11. The method of claim 1, wherein the subject is human.

12. The method of claim 1, wherein the stimulus is thermal.

13. The method of claim 1, further comprising measuring another indicator of pain, wherein the indicator is verbal or nonverbal.

14. A method of determining efficacy of an analgesic in a subject comprising:
a. administering the analgesic to a subject;
b. applying a stimulus to the subject;
c. measuring brain activity of the subject in response to the stimulus using fMRI and generating a brain map of the subject representing the brain activity of the subject;
d. comparing the brain map of the subject to a signature map indicative of pain to determine the difference between the brain map of the subject and the signature map,
wherein the signature map represents brain activity indicative of pain, and wherein the dissimilarity between the brain map of the subject and the signature map is indicative of the efficacy of the analgesic.

15. The method of claim 14, wherein the signature map comprises a fMRI pattern that is at least 70% identical to the fMRI pattern shown in FIG. 1A.

16. The method of claim 14, wherein the analgesic is administered before, after or concurrently with the stimulus.

17. A method to diagnose a pain-related condition comprising:
a. measuring brain activity of a subject using fMRI and generating a brain map of the subject representing the brain activity of the subject; and
b. comparing the brain map of the subject to a signature map to determine the functional connectivity or structural connectivity between the brain regions of the subject;
wherein the signature map represents brain activity indicative of pain.

18. The method of claim 17, wherein the signature map comprises a fMRI pattern that is at least 70% identical to the fMRI pattern shown in FIG. 1A.

19. (canceled)

20. (canceled)

21. (canceled)
